(19) United States
(12) Patent Application Publication
(10) Pub. No.: US 2023/0265123 A1
(43) Pub. Date: Aug. 24, 2023

(54) PEPTIDES AND FORMULATIONS FOR CANCER TREATMENT

(71) Applicant: Peptinovo Biopharma, Inc., Ann Arbor, MI (US)

(72) Inventor: Reynold Homan, Ann Arbor, MI (US)

(21) Appl. No.: 18/041,289

(22) PCT Filed: Aug. 10, 2021
(86) PCT No.: PCT/US2021/045337

§ 371 Date: Feb. 10, 2023

Related U.S. Application Data
(60) Provisional application No. 63/064,288, filed on Aug. 11, 2020.

(51) Int. Cl. C07K 7/08; A61K 9/51; A61P 35/00; A61K 47/54; A61K 51/08; A61K 51/04

(52) U.S. Cl. CPC C07K 7/08; A61K 9/5123; A61P 35/00; A61K 47/554; A61K 47/542; A61K 51/08; A61K 51/0408

(57) ABSTRACT

Novel amphiphilic peptide, peptide amphiphile lipid micelles, processes for making peptide amphiphile lipid micelles comprising an amphiphilic peptide and phospholipid and optionally comprising a cargo molecule, and methods of use.

### CROSS REFERENCE TO RELATED APPLICATIONS

This PCT application claims the Benefit of U.S. Provisional Application Ser. No. 63/064,288, filed Aug. 11, 2020. The disclosure of which is hereby incorporated by reference in its entirety.

### SEQUENCE LISTING

This application incorporates by reference in its entirety the sequence listing entitled “236603_496246_Sequence_Listing_ST25.txt” created on Aug. 2, 2021, at 2:11 PM, that is 6 KB, and filed electronically herewith.

### FIELD

The present invention relates to the transport and delivery of therapeutic molecules to their sites of action via parenteral administration. More particularly, the present invention concerns a formulation technology enabling the incorporation of drugs into nanoparticles that can be readily administered parenterally for the safe and effective delivery of the incorporated drugs to their therapeutic targets.

### BACKGROUND

Options for dealing with hydrophobic drugs requiring parenteral dosing usually involve the addition of various excipients to obtain stable suspensions, dispersions, or solutions suitable for injection. The types of excipients used include detergents, polymers of various types, oil emulsions, phospholipid, and albumin. In some cases, the excipients used to obtain the necessary drug solubilization are detergent-like substances. These include deoxycholate; Cremophor EL®, a polyethyloxated derivative of castor oil; and polysorbate 80. The latter two are typically used in tandem with ethanol. These agents solve the solubilization problem but they have noxious properties which introduce a high risk of hypersensitivity reactions. It is a common requirement that patients injected with solutions containing Cremophore EL® or polysorbate 80 are pretreated with anti-inflammatory drugs to subdue formulation-dependent inflammation. The most serious consequences of hypersensitivity reactions are reduced tolerance to treatment and increased risk of death. Recently, U.S. Pat. No. 10,532,105, was issued and covers different peptides and formulations from the presently filed application. Given the limited number of current treatments, there is a clear need to provide formulation alternatives for parenterally administered drugs with improved safety profiles and therapeutic indexes, and/or therapies which at least provide the public with a useful choice.

### SUMMARY

The present disclosure addresses this need by providing novel nanoparticle formulations of lipid and peptide and methods to form them that allow incorporation of molecules, e.g., drugs and are stable in infusion or injection solutions. The formulations of the invention provide one or more improvements, including but not limited to, improved pharmacokinetic parameters, increased half-life, targeted delivery, diminished toxicity or an improved therapeutic index for parenterally administered drugs, particularly for, anti-cancer drugs.

The present disclosure provides an amphiphilic, alpha-helical peptide that comprises an amino acid sequence of SEQ ID NO:7, SEQ ID NO:9, or SEQ ID NO:10.

Further, the present disclosure provides peptide-amphiphile lipid micelles (PALM) which comprise a peptide comprising an amino acid sequence of the disclosure, sphingomyelin and one or more additional phospholipids. The PALM of the present disclosure optionally comprise one or more cargo molecules, such as imaging agents and drugs.

The present disclosure also provides for processes for preparing PALM and PALM composition formulated with cargo molecules.

Additionally, the present disclosure provides for compound conjugates and methods of preparing compound conjugates suitable for use with PALM.

Further the present disclosure provides for methods of treating disorders by administering PALM-drug conjugates.

### BRIEF DESCRIPTION OF THE FIGURES

FIG. 1. The size exclusion chromatogram of PALM containing miriplatin (solid line) compared to human HDL(dashed line). PALM was composed of a SEQ ID NO:1 peptide with POPC, SM and miriplatin at a 2.5:3:7:0.75 mole ratio.

FIG. 2. The size exclusion chromatograph of PALM containing XC and prepared with the peptide of SEQ ID NO:1 at a peptide:POPC:SM:XC mole ratio of 1:2.8:1.2:0.4. The elution positions of protein standards of various Stokes diameters are marked.

FIG. 3. Comparison of the size exclusion chromatograms for PALM containing XT3 and prepared with the peptide of SEQ ID NO:1 (dashed line) or with R4F peptide (solid line). The composition of both was peptide:POPC:SM:XT3 at a mole equivalent ratio of 1:2.8:1.2:0.4.

FIG. 4. Depicts the size exclusion chromatogram of PALM prepared with a SEQ ID NO:1 peptide and containing fenretinide. The PALM composition was peptide:POPC:SM:fenretinide at a mole equivalent ratio of 2.5:3:7:2.

FIG. 5. Inhibition of PC3 prostate cancer cell growth by miriplatin in PALM compared to inhibition by cisplatin. PALM was composed of 1 mole equivalent SEQ ID NO:1 peptide with 4 mole equivalents of a 3:7 mole ratio of POPC and SM with 0.3 mole equivalents of miriplatin. The lines indicate the fit of the data to the logistic function.

FIG. 6. Inhibition of SKOV3 ovarian cancer cell growth after 72 hours incubation with PALM(XC) (square, dotted line) or PALM(XT3) (diamond, solid line) compared to paclitaxel (circle, dashed line). Percent growth was determined by MTT assay. The lines are fits of the data to the logistic equation.

FIG. 7. Effect of PALM peptide on uptake of DiI fluorescent dye from PALM by BHK cells bearing a mifepristone-inducible, human scavenger receptor B, type I gene. DiI uptake tested with cells in the uninduced, basal state (open bar), or after mifepristone induction (filled bar). Human HDL, containing DiI, was tested for reference. The amount of DiI taken up by cells over 4 hours of incubation was detected by fluorescence.

FIG. 8. Inhibition of SKOV3 cell growth by PALM(XT3), containing the peptide of SEQ ID NO:1, is blocked by SR-BI antibody (arrow).

FIG. 9. SKOV3 tumor growth inhibition in female, athymic mice dosed with Taxol vehicle (circle), 10 mg/kg Taxol (square), 8 mg/kg paclitaxel equivalents as PALM(XT3) (inverted triangle), 24 mg/kg paclitaxel equivalents as PALM(XT3) (diamond), and PALM alone, without XT3 (triangle). Seven days after SKOV3 tumor initiation, mice were given 6 intravenous doses spaced at 4-day intervals. Tumor volumes were measured 15 days after the last dose. p<0.01 vs. vehicle (*), vs. Taxol (#), vs. XT3-8 (‡). PALM contains a SEQ ID NO:5 peptide.

FIG. 10. Growth inhibition of human triple-negative breast cancer cells (SUM185, BT549, MDA-MB231) and human ovarian cancer cells (0V90) by cholesteryl (N4)-gemcitabine carbamate loaded in PALM. The percent growth relative to untreated cells was determined by MTT assay. The lines are fits of the data to the logistic equation.

### DETAILED DESCRIPTION

### Definitions

“Nanoparticle” means a particle having no dimension greater than 100 nm.

As used herein, the singular forms “a”, “an” and “the” include plural references unless the context clearly dictates otherwise.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms” consists of” and “consisting of” have the meaning ascribed to them in United States Patent law; namely that these terms are close ended.

The antecedent “about” indicates that the values are approximate. For example, the range of “about 1 mg to about 50 mg” indicates that the values are approximate values. The range of “about 1 mg to about 50 mg” includes approximate and specific values, e.g., the range includes about 1 mg, 1 mg, about 50 mg and 50 mg.

When a range is described, the range includes both the endpoints of the range as well as all numbers in between. For example, “between 1 mg and 10 mg” includes 1 mg, 10 mg and all amounts between 1 mg and 10 mg. Likewise, “from 1 mg to 10 mg” includes 1 mg, 10 mg and all amounts between 1 mg and 10 mg.

As used herein, “alkyl” refers to a saturated aliphatic hydrocarbon group containing from 7-21 carbon atoms. As used herein, the terminology (C₁-Cn) alkyl refers to an alkyl group containing 1-n carbon atoms. For example, (C₈-C₁₂) alkyl refers to an alkyl group containing 8, 9, 10, 11, or 12 carbon atoms. An alkyl group can be branched or unbranched.

As used herein, “alkenyl” refers to an aliphatic carbon group that contains from 7-21 carbon atoms and at least one double bond. As used herein, the terminology (C₁-Cₙ) alkenyl refers to an alkenyl group containing 1-n carbon atoms. An alkenyl group can be branched or unbranched.

“Effective amount” or a “pharmaceutically-effective amount” in reference to the composition containing PALM, refers to the amount of said composition sufficient to induce a desired biological, pharmacological, or therapeutic outcome in a subject.

“Consisting essentially of” when used to describe the lipid component means that the lipid component includes less than 0.1 mol % of any additional lipid other than those specified.

“XC” is an abbreviation for paclitaxel 2′-cholesteryl carbonate.

“XT3” is an abbreviation for paclitaxel 2′-δ-tocotrienyl carbonate.

“CGC” is an abbreviation for cholesteryl (N4)-gemcitabine carbamate.

“C₁₆PTX” is an abbreviation for paclitaxel 2′-palmitate.

“MP” is an abbreviation for miriplatin.

“PTX” is an abbreviation for paclitaxel

“POPC” is an abbreviation for 1-palmitoyl-2-oleoyl phosphatidylcholine

“SM” is an abbreviation for sphingomyelin

“HDL” is an abbreviation for high density lipoprotein.

“SR-BI” is an abbreviation for scavenger receptor class B, type 1.

“BHK” is an abbreviation for baby hamster kidney.

“DiI” is an abbreviation for 1,1′-dioctadecyl-3,3,3′,3′-tetramethylindocarbocyanine.

“MTT” is an abbreviation for thiazolyl blue tetrazolium bromide.

“PBS” is an abbreviation for Dulbecco's phosphate-buffered saline.

“PALM” is an acronym used to identify the peptide-amphiphile lipid micelles formed from a combination of amphiphilic peptide with phospholipids and optionally other hydrophobic molecules, in aqueous suspension.

“Amphiphilic” describes a molecule or polymer (e.g. peptide) with affinity for both lipid and aqueous phases due to a conformation in which hydrophilic (water seeking) substituents and hydrophobic (water avoiding) substituents in the molecule or polymer are structurally segregated from one another.

“Lipophilic” describes a substance that distributes preferentially to lipid domains of lipid-rich particles in aqueous suspension. The lipid-rich particles include lipid micelles, liposomes, lipoproteins, cell membranes and lipid emulsions.

“Peptide” is a polymer produced from alpha-amino acid monomers joined together by amide bonds formed between the carboxylic group of one amino acid and the alpha-amine group of the next amino acid in the polymer. “Peptide” also includes a polymer of amino acid monomers joined together. Both L-optical isomers and the D-optical isomers of amino acids can be used. Amino acids making up the polymer may be either those found in nature (i.e. natural amino acids) or un-natural amino acids. The term “residue” or “amino acid residue” includes reference to an amino acid that is incorporated into a peptide, polypeptide, or protein.

Peptide sequences according to convention, and as used herein, are written N-terminus to C-terminus, left to right.

“Micelle” is a multi-molecular structure organized by non-covalent interactions in an aqueous phase. The micelle is composed of amphiphilic and hydrophobic molecules which aggregate in such a manner that the hydrophobic domains of molecules are shielded from the water and the hydrophilic constituents are at the micelle-water interface.

“Cargo molecules” are hydrophobic or amphiphilic molecules with pharmaceutical, therapeutic, or diagnostic properties that are stably incorporated into PALM and do not disrupt the stability of PALM.

“Aib” is the three letter code for the amino acid α-methyl alanine, alternately, α-amino isobutyric acid.

“Aba” is the three letter code for the amino acid alpha-amino butyric acid.

“Amy” is the three letter code for the amino acid alpha-methyl L-valine.

“Aml” is the three letter code for the amino acid α-methyl L-leucine.

“Amp” is the three letter code for the amino acid α-methyl L-phenylalanine.

“Amt” is the three letter code for the amino acid α-methyl L-tryptophan.

“Orn” is the three letter code for the amino acid ornithine.

“SEC” is size exclusion chromatography

“DLS” is dynamic light scattering

A first aspect of the present disclosure provides “amphiphilic peptides”. Amphiphilic peptides are able to adopt an alpha helical conformation in which the helix has opposing polar and non-polar faces oriented along the long axis of the helix. Techniques of synthesizing peptides are well known in the art. The peptides of the present disclosure can be synthesized by any technique known in the art.

Table 1 shows the charge distribution of specific amphiphilic peptides of the present disclosure compared with several prior art sequences. The charge distribution of the peptides of the present invention are novel in view of the prior art shown below.

TABLE 1
Charges of Residues in Apolipoprotein A-I Mimetic Peptides at Neutral pH

| | N-Term | Amino Acid Position | | | | | | | | | | | | | | | | | | | | | | | | C-Term |
|---|---|---|---|---|---|---|---|---|---|---|---|---|---|---|---|---|---|---|---|---|---|---|---|---|---|---|
| Peptide | Charge | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | Charge |
| SEQ ID^{a} | + | − | ∘ | ∘ | ∘ | + | ∘ | ∘ | − | ∘ | ∘ | ∘ | ∘ | ∘ | ∘ | − | + | ∘ | + | ∘ | ∘ | | | | | − |
| A-I_{con}^{b} | + | ∘ | ∘ | ∘ | − | − | ∘ | + | − | + | ∘ | ∘ | − | ∘ | ∘ | − | ∘ | ∘ | + | ∘ | + | ∘ | + | | | − |
| LAP642^{c} | + | ∘ | ∘ | ∘ | − | ∘ | ∘ | + | − | ∘ | ∘ | ∘ | − | ∘ | ∘ | − | ∘ | ∘ | + | ∘ | + | ∘ | + | | | − |
| 18A^{d} | + | − | ∘ | ∘ | + | ∘ | ∘ | ∘ | − | + | ∘ | ∘ | − | + | ∘ | + | − | ∘ | ∘ | | | | | | | − |
| 2F^{e} | ∘ | − | ∘ | ∘ | + | ∘ | ∘ | ∘ | − | + | ∘ | ∘ | − | + | ∘ | + | − | ∘ | ∘ | | | | | | | ∘ |
| R4F^{f} | ∘ | ∘ | ∘ | − | + | ∘ | + | − | ∘ | ∘ | + | − | ∘ | ∘ | ∘ | + | ∘ | ∘ | − | | | | | | | ∘ |
| FAMP^{g} | + | ∘ | ∘ | − | ∘ | ∘ | ∘ | ∘ | ∘ | ∘ | − | + | ∘ | ∘ | + | ∘ | ∘ | − | − | ∘ | ∘ | + | + | ∘ | ∘ | − |

^{a}SEQ ID NO: 1
^{b}Anantharamaiah et al. (1990) Arteriosclerosis 10: 95-105
^{c}Homan et al. (2013) Anal. Biochem. 441: 80-86
^{d}Anantharamaiah et al. (1985) J. Biol. Chem. 260: 10248-10255
^{e}Datta et al. (2001) J. Lipid Res. 42: 1096-1104
^{f}Zhang et al. (2009) Angew. Chem. Int. Ed. 48: 9171-9175
^{g}Uehara et al. (2013) J Am Heart Assoc. 2(3): e000048. doi: 10.1161/JAHA.113.000048
“∘” indicates zero charge at the indicated position.
“+” indicates a positive charge at the indicated position.
“−” indicates a negative charge at the indicated position.

Also according to the first aspect of the disclosure, any peptide disclosed in the present invention may include from 1-4 additional amino acids independently added to either the N-terminus or C-terminus of the amino acid sequence. The additional amino acids are selected such that the addition of the amino acids does not negatively affect the amphiphilicity of the peptide. It is contemplated that any of the disclosed embodiments of the peptides according to the first aspect are optionally acylated at the alpha-amine of the N-terminal amino acid of the peptide, optionally amidated at the terminal carboxyl group of the peptide, or optionally acylated at the alpha-amine of the N-terminal amino acid and amidated at the terminal carboxyl group of the peptide. Peptides can be acylated or amidated by methods known in the art.

One embodiment of the first aspect of the disclosure provides a peptide that comprises the amino acid sequence: X1-X2-X3-X4-X5-X6-X7-X8-X9-X10-X11-X12-X13-X14-X15-X16-X17-X18-X19-X20, wherein: X1 is D; X2 is V, Aib, Amv, or Aml; X3 and X10 are each F; X4 and X19 are each Q; X5, X16, and X18 are each K; X6, X9, and X13 are each L; X7 and X14 are each independently selected from the group consisting of Aib, Amy, Aml, Amp, Amt; X8 and X15 are each E; X11 and X12 are each independently selected from the group consisting of Q and N; X17 is W; and X20 is V, (SEQ ID NO:12) wherein the peptide is from 20 to 24 amino acids in length.

Another embodiment of the first aspect of the disclosure provides a peptide that consists essentially of the amino acid sequence: X1-X2-X3-X4-X5-X6-X7-X8-X9-X10-X11-X12-X13-X14-X15-X16-X17-X18-X19-X20, wherein: X1 is D; X2 is V, Aib, Amy, or Aml; X3 and X10 are each F; X4 and X19 are each Q; X5, X16, and X18 are each K; X6, X9, and X13 are each L; X7 and X14 are each independently selected from the group consisting of Aib, Amv, Aml, Amp, Amt; X8 and X15 are each E; X11 and X12 are each independently selected from the group consisting of Q and N; X17 is W; and X20 is V; (SEQ ID NO:12) wherein the peptide is from 20 to 24 amino acids in length.

In yet another embodiment of the first aspect of the disclosure provides a peptide that consists of the amino acid sequence: X1-X2-X3-X4-X5-X6-X7-X8-X9-X10-X11-X12-X13-X14-X15-X16-X17-X18-X19-X20, wherein: X1 is D; X2 is V, Aib, Amv, or Aml; X3 and X10 are each F; X4 and X19 are each Q; X5, X16, and X18 are each K; X6, X9, and X13 are each L; X7 and X14 are each independently selected from the group consisting of Aib, Amy, Aml, Amp, Amt; X8 and X15 are each E; X11 and X12 are each independently selected from the group consisting of Q and N; X17 is W; and X20 is V (SEQ ID NO:12).

It is contemplated that any of the disclosed embodiments of the peptides according to the first aspect are optionally acylated at the alpha-amine of the N-terminal amino acid of the peptide, optionally amidated at the terminal carboxyl group of the peptide, or optionally acylated at the alpha-amine of the N-terminal amino acid and amidated at the terminal carboxyl group of the peptide. Peptides can be acylated or amidated by methods known in the art.

Another embodiment of the first aspect of the disclosure of SEQ ID NO:7. Yet another embodiment of the first aspect of the disclosure of SEQ ID NO:9. Another embodiment of the first aspect of the disclosure of SEQ ID NO:10.

Particular peptides of the present invention are provided in Table 2.

TABLE 2

| SEQ ID NO: | Peptide Sequence¹ |
|---|---|
| 7 | D{Aib}FQKL{Aib}ELFNQL{Aib}EKWKQV |
| 9 | DVFQKL{Aib}ELFQQL{Aib}EKWKQV |
| 10 | DVFQKL{Aib}ELFQNL{Aib}EKWKQV |

A second aspect of the disclosure provides peptide amphiphile lipid micelles (PALM) formed from a combination of amphiphilic peptide with phospholipids. PALM of the second aspect of the disclosure comprises one or more peptides of the first aspect of the disclosure complexed with a lipid component where the lipid component comprises sphingomyelin and one or more additional phospholipids. PALM according to the present disclosure may be passively or actively delivered to a target cell population. In one embodiment of the second aspect of the disclosure, PALM comprises one or more peptides of the present disclosure where the lipid component consists essentially of sphingomyelin and one or more additional phospholipids. In one embodiment PALM comprises a peptide of the present disclosure and a lipid component wherein the lipid component comprises sphingomyelin and one or more additional phospholipids where the additional phospholipid is selected from the group consisting of phosphatidylcholine, polyethylene glycol-phosphatidylethanolamine (PEG-PE), phosphatidylethanolamine, phosphatidylglycerol, phosphatidylserine, phosphatidylinositol, cardiolipin, and any combination thereof. In another embodiment the PALM comprises a peptide of the disclosure and the lipid component comprises sphingomyelin, and phosphatidylcholine. In another embodiment the PALM comprises a peptide of the disclosure, sphingomyelin, and 1-palmitoyl-2-oleoyl phosphatidylcholine (POPC). In yet another embodiment the PALM comprises a peptide of the disclosure and the lipid component comprises sphingomyelin, and phosphatidylethanolamine. In yet another embodiment the PALM comprises a peptide of the disclosure, and the lipid component comprises sphingomyelin, and poly(ethylene glycol)phosphatidyl-ethanolamine. In still another embodiment the PALM comprises a peptide of the disclosure and the lipid component comprises sphingomyelin, and phosphatidylserine. In another embodiment the PALM comprises a peptide of the disclosure and the lipid component comprises sphingomyelin and cardiolipin. In another embodiment of the of the second aspect of the disclosure the PALM comprises peptide of the disclosure and the lipid component of comprises phosphatidylcholine and one or more additional phospholipids. In another embodiment the lipid component comprises 1-palmitoyl-2-oleoyl phosphatidylcholine (POPC) and one or more additional phospholipids.

In still another embodiment of the second aspect of the disclosure, PALM comprises a peptide of the disclosure and the lipid component consists essentially of sphingomyelin and one or more additional phospholipids where the one or more additional phospholipids is selected from the group consisting of phosphatidylcholine, polyethylene glycol-phosphatidyl-ethanolamine (PEG-PE), phosphatidylethanolamine, phosphatidylglycerol, phosphatidylserine, phosphatidylinositol, cardiolipin, and any combination thereof. In still another embodiment the PALM comprises a peptide of the disclosure and the lipid component consists essentially of sphingomyelin and phosphatidylcholine. In another embodiment the PALM comprises a peptide of the disclosure and the lipid component consists essentially of sphingomyelin and 1-palmitoyl-2-oleoyl-phosphatidylcholine (POPC). Or in still another embodiment of the second aspect of the disclosure, PALM comprises a peptide of the disclosure and the lipid component consists essentially of phosphatidylcholine and one or more additional phospholipids. In another embodiment the lipid component comprises 1-palmitoyl-2-oleoyl phosphatidylcholine (POPC) and one or more additional phospholipids.

In some embodiments of the second aspect of the disclosure, PALM comprises a peptide of the disclosure and the lipid component comprises sphingomyelin and one or more additional phospholipids where the one or more additional phospholipid is selected from the group consisting of phosphatidylcholine, polyethylene glycol-phosphatidylethanolamine (PEG-PE), phosphatidylethanolamine, phosphatidylglycerol, phosphatidylserine, phosphatidylinositol, cardiolipin, and any combination thereof where the molar ratio of phospholipid to sphingomyelin is from about 95:5 to about 5:95. In another embodiment the molar ratio of phospholipid to sphingomyelin is from about 95:5 to about 10:90. In another embodiment the molar ratio of phospholipid to sphingomyelin is from about 90:10 to about 20:80. In still another embodiment the molar ratio of phospholipid to sphingomyelin is from about 25:75 to about 35:65. In another embodiment the molar ratio of phospholipid to sphingomyelin is about 30:70. In another embodiment the molar ratio of phospholipid to sphingomyelin is from about 80:20 to about 60:40. In yet another embodiment the molar ratio of phospholipid to sphingomyelin is from about 75:25 to about 65:35. In still another embodiment the molar ratio of phospholipid to sphingomyelin is about 70:30.

The fatty acid constituents of the phospholipids include fatty acids according to the formula: R—COOH, wherein R is a (C₇-C₂₁) alkyl group or a (C₇-C₂₁) alkenyl group wherein the alkenyl group can have from one to six double bonds. Examples of suitable fatty acids include, but are not limited to, phytanic acid, linolenic acid, linoleic acid, docosatetraenoic acid, oleic acid, caprylic acid, lauric acid, arachidic acid, myristic acid and palmitic acid. The pair of fatty acids esterified to the glycerol backbone of a particular phospholipid may be identical or each may be a different type of fatty acid.

The molar ratio of the lipid component to peptide is from about 10:1 to about 2:1. In one embodiment the ration is from about 9:1 to about 2:1. In one embodiment the molar ratio of the lipid component to peptide is from about 8:1 to about 2:1. In still another embodiment the molar ratio of the lipid component to peptide is from about 7:1 to about 3:1. In another embodiment the molar ratio of the lipid component to peptide is from about 6:1 to about 4:1.

Complexes of phosphatidylcholine with amphiphilic peptides are known. One method to produce these complexes is by initial co-lyophilization from a common solvent phase followed by rehydration of the dry lyophilizate to form complexes in aqueous suspension.

Particle size is measured by DLS and is expressed as the hydrodynamic mean diameter (“mean diameter”). PALM according to the second aspect of the disclosure are nanometer-sized particles having a mean particle diameter of 100 nm or less, 50 nm or less, 40 nm or less, or 30 nm or less. In one embodiment the mean particle diameter is from about 5 nm to about 100 nm. In another embodiment the mean particle diameter is from about 5 nm to about 50 nm. In yet another embodiment the mean particle diameter is from about 5 nm to about 40 nm. In one embodiment the mean particle diameter is from about 5 nm to about 30 nm. In yet another embodiment the mean particle diameter is from about 7.5 nm to about 30 nm. In still another embodiment the mean particle diameter is from about 10 nm to about 30 nm. In another embodiment the mean particle diameter is from about 5 nm to about 25 nm. In another embodiment the mean particle diameter is from about 7.5 nm to about 25 nm. In yet another embodiment the mean particle diameter is from about 10 nm to about 25 nm. In another embodiment the mean particle diameter is from about 5 nm to about 20 nm. In another embodiment the mean particle diameter is from about 7.5 nm to about 20 nm. In yet another embodiment the mean particle diameter is from about 10 nm to about 20 nm. In still another embodiment the mean particle diameter is from about 5 nm to about 15 nm. In another embodiment the mean particle diameter is from about 7.5 nm to about 15 nm. In yet another embodiment the mean particle diameter is from about 10 nm to about 15 nm. In still another embodiment the mean particle diameter is from about 7.5 nm to about 10 nm.

A third aspect of the disclosure provides for PALM-cargo molecule compositions which comprise any one of the PALM embodiments of the second aspect of the disclosure and a cargo molecule. Cargo molecules include, but are not limited to, molecules having pharmaceutical or therapeutic properties. Non-limiting examples of cargo molecules include anti-cancer compounds such as all-trans retinoic acid, alcohol esters of all-trans retinoic acid including methyl-, ethyl-, and longer chain fatty alkyl chain alcohol esters of retinoic acid and cholesteryl esters of retinoic acid; retinoic acid amides such as fenretinide; retinol and carboxylic acid esters of retinol including methyl-, ethyl-, and longer chain fatty alkyl chain alcohol esters of retinoic acid; lipophilic anti-fungal agents such as amphotericin B or nystatin; steroids such as progesterone, testosterone, prednisolone, hydrocortisone, dexamethasone and estradiols; analgesics such as propofol and haloperidol; antipsychotics such as fluphenazine decanoate and aripiprazole; the vitamin D analogs cholecalciferol and ergocalciferol; and the isomers of vitamin E, either collectively or individually.

Cargo molecules also include molecules enabling diagnostic or imaging procedures such as fluorescent imaging agents, radiolabeled imaging agents, and agents used for MRI, PET, CT, SPECT/CT and x-ray studies. MRI imaging agents include, but are not limited to, contrast agents such as a phosphatidylethanolamine with a diethylenetriamine pentaacetic acid moiety that is chelated with a gadolinium ion or similar lanthanide ion or indium-111 or gallium-67 or lutetium-177 or samarium-153.

Cargo molecules may also be various types and lengths of RNA or DNA that have been linked to cholesterol or other polycyclic fatty alcohols by known methods.

In one embodiment of the third aspect, the cargo molecule is miriplatin which has the chemical name: cis-[((1R, 2R)-1,2-cyclohexanediamine-N,N)bis(myristato)] platinum(II).

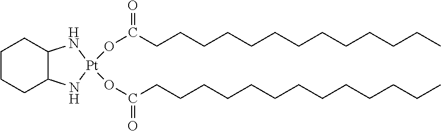

Yet another embodiment of the third aspect of the disclosure, is a PALM-cargo molecule complex wherein the cargo molecule is a compound conjugate of formula I

A-R-L-X (formula I)

wherein A is an agent having a hydroxy or amine group; R is a hydroxyl or an amine group of the agent; L is a linker, and X is an anchor moiety.

Another embodiment of the third aspect of the disclosure is a PALM-cargo molecule complex wherein the cargo molecule is a compound conjugate of formula I:

A-R-L-X (formula I)

wherein A is an agent having a hydroxy or amine group; R is the hydroxyl or the amine group of the agent; L is carbonic acid, succinic acid or diglycolic acid; and X is cholesterol, α-tocotrienol, β-tocotrienol, γ-tocotrienol, δ-tocotrienol, cholesterol, coprostanol, plant sterols, (β-sitosterol, sitostanol, stigmasterol, stigmastanol, campesterol, brassicasterol), ergosterol, retinol, cholecalciferol, ergocalciferol, tocopherol, or tocotrienol.

Another embodiment of the third aspect of the disclosure is a PALM-cargo molecule complex wherein the cargo molecule is a compound conjugate of formula I:

wherein A is an agent having a hydroxy or amine group; R is the hydroxyl or the amine group of the agent; L is selected from the group consisting of carbonic acid, succinic acid or diglycolic acid; and X is selected from the group consisting of cholesterol, α-tocotrienol, β-tocotrienol, γ-tocotrienol, δ-tocotrienol, cholesterol, coprostanol, plant sterols, (β-sitosterol, sitostanol, stigmasterol, stigmastanol, campesterol, brassicasterol), ergosterol, retinol, cholecalciferol, ergocalciferol, α-tocopherol, β-tocopherol, γ-tocopherol, and δ-tocopherol,

Another embodiment of the third aspect of the disclosure is a PALM-cargo molecule complex wherein the cargo molecule is a compound conjugate of formula I:

wherein A is an agent having a hydroxy or amine group; R is a hydroxyl or an amine group of the agent; L is a linker; and X is an anchor moiety selected from the group consisting of cholesterol, cholecalciferol and δ-tocotrienol.

In one embodiment of a compound conjugate of formula (1), R is a hydroxy group of the agent, and the anchor moiety is covalently bonded to agent by a carbonate ester bond. In another embodiment of a compound conjugate of formula (1), R is an amine group of the agent, and the anchor moiety is covalently bonded to agent by a carbamate ester bond.

In another embodiment of a compound conjugate of formula (1), the anchor moiety is cholesterol. In still another embodiment of a compound conjugate of formula (1), the anchor moiety is cholesterol, with the proviso that if the anchor moiety is cholesterol, then the compound is not paclitaxel.

In yet another embodiment of a compound conjugate of formula (1) the anchor moiety is α-tocotrienol. In another embodiment of a compound conjugate of formula (1) the anchor moiety is β-tocotrienol. In still another embodiment of a compound conjugate of formula (1) the anchor moiety is γ-tocotrienol. In yet another embodiment of a compound conjugate of formula (1) the anchor moiety is δ-tocotrienol. In still another embodiment of a compound conjugate of formula (1) the anchor moiety is ergocalciferol.

In some embodiments of the compound conjugate of formula (1) the agent is a drug. In some embodiments of the compound conjugate of formula (1) the agent is an anti-cancer drug. In one embodiment of the compound conjugate of formula (1) the agent is an anti-cancer drug and the anti-cancer drug is covalently bonded to the anchor by a carbonate ester bond. In one embodiment of the compound conjugate of formula (1) the agent is an anti-cancer drug and the anti-cancer drug is covalently bonded to the anchor by a carbamate ester bond.

Still other embodiments include anti-cancer drugs having a hydroxyl group available to form the carbonate ester bond, for example, one or more of AZD2811, a hydroxy camptothecin, cabazitaxel, doxorubicin, epothilone B, eribulin, ixabepilone, troxacitabine, vincristine, sirolimus, tubulysin A, docetaxel, or paclitaxel.

In other embodiments include anti-cancer drugs having an amine available to form the carbamate ester bond, for example, one or more of doxorubicin, daunorubicin, gemcitabine, cytarabine and troxacitabine.

In another embodiment, chemotherapeutic agents are bortezomib, carboplatin, cisplatin, gemcitabine, misonidazole, oxaliplatin, procarbazine, thalidomide, docetaxel, hexamethylmelamine, paclitaxel, vincristine, vinblastine, or vinorelbine.

In one embodiment of the invention, the chemotherapeutic agent is docetaxel, paclitaxel, carboplatin, doxorubicin, topotecan, irinotecan, cyclophosphamide, cisplatin, gemcitabine, cyclophosphamide, oxaliplatin, capecitabine, 5-fluorouracil and leucovorin.

In some embodiments of the PALM-cargo molecule compositions of the third aspect of the disclosure, the cargo molecule is paclitaxel 2′-cholesteryl carbonate. In another embodiment the cargo molecule is paclitaxel 2′-δ-tocotrienyl carbonate.

In yet other embodiments, the PALM-cargo molecule is docetaxel 2′-cholesteryl carbonate. In other embodiments, the cargo molecule is the cholesteryl carbonate ester of 10-hydroxycamptothecin. In still other embodiments, the cargo molecule is the cholesteryl carbonate ester of 7-ethyl-10-hydroxycamptothecin, which is the active metabolite of irinotecan. In another embodiment, the cargo molecule is the cholesteryl carbonate ester of sirolimus. In other embodiments, the cargo molecule is the cholesteryl carbamate ester of gemcitabine. In other embodiments, the cargo molecule is the cholesteryl carbonate ester of tubulysin A. In other embodiments, the cargo molecule is a cholesteryl carbonate ester of morphine. In other embodiments, the cargo molecule is a cholesteryl carbonate ester of hydromorphone. In yet another embodiment, the cargo molecules is a cholesteryl carbonate ester of codeine.

In other embodiments of the PALM-cargo molecule compositions of the third aspect of the disclosure, the cargo molecule is the cholesteryl carbamate ester of gemcitabine (Cholesteryl (N⁴)-Gemcitabine Carbamate). In other embodiments, the cargo molecule is the cholesteryl carbamate ester of adenosine. In yet other embodiments, the cargo molecule is the cholesteryl carbonate ester of adenosine.

In yet other embodiments, the cargo molecule is the cholesteryl carbonate ester of doxorubicin, the structure of which is:

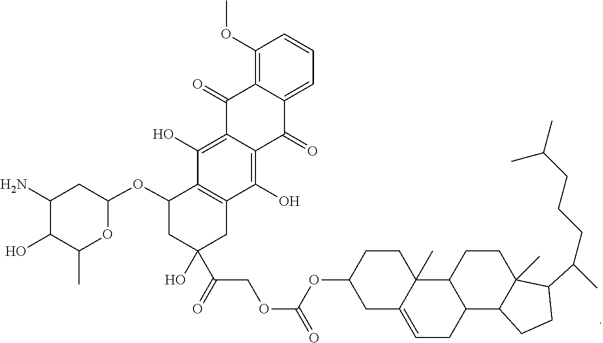 [SMILES: COc1cccc2c1C(=O)c1c(O)c3c(c(O)c1C2=O)CC(O)(C(=O)COC(=O)OC1CCC2(C)C(=CCC4C2CCC2(C)C(C(C)CCCC(C)C)CCC42)C1)CC3OC1CC(N)C(O)C(C)O1]

In yet another embodiment, the cargo molecule is the cholesteryl carbonate ester of vincristine, the structure of which is:

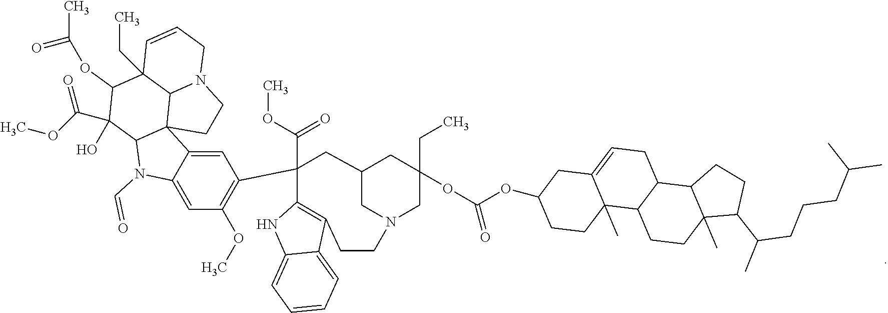 [SMILES: CCC1(OC(=O)OC2CCC3(C)C(=CCC4C3CCC3(C)C(C(C)CCCC(C)C)CCC43)C2)CC2CN(CCc3c([nH]c4ccccc34)C(C(=O)OC)(c3cc4c(cc3OC)N(C=O)C3C(O)(C(=O)OC)C(OC(C)=O)C5(CC)C=CCN6CCC43C65)C2)C1]

In still another embodiment the cargo molecule is the delta-tocotrienyl carbonate ester of paclitaxel, the structure of which is:

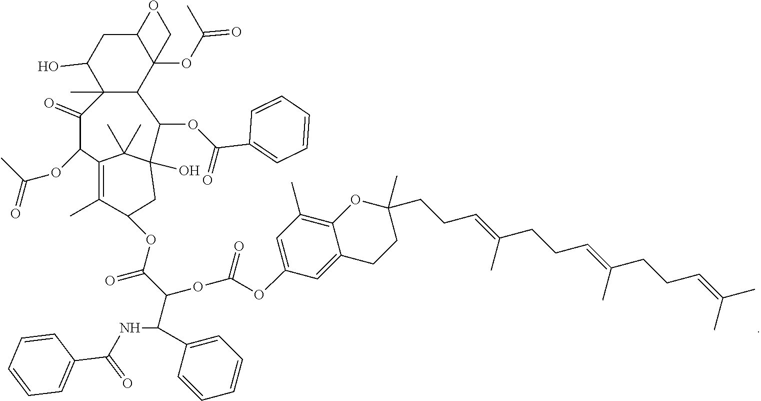 [SMILES: CC(=O)OC1C(=O)C2(C)C(O)CC3OCC3(OC(C)=O)C2C(OC(=O)c2ccccc2)C2(O)CC(OC(=O)C(OC(=O)Oc3cc(C)c4c(c3)CCC(C)(CC/C=C(\C)CC/C=C(\C)CCC=C(C)C)O4)C(NC(=O)c3ccccc3)c3ccccc3)C(C)=C1C2(C)C]

In still another embodiment the cargo molecule is the gemcitabine delta-tocotrienlyl carbamate ester, the structure of which is:

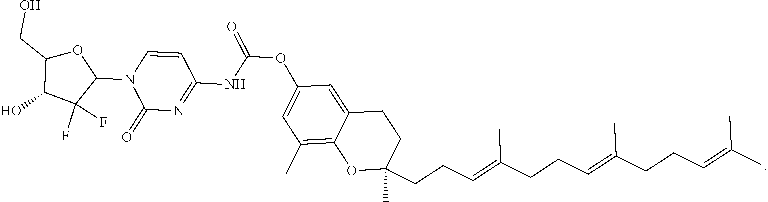 [SMILES: CC(C)=CCC/C(C)=C/CC/C(C)=C/CC[C@]1(C)CCc2cc(OC(=O)Nc3ccn(C4OC(CO)[C@@H](O)C4(F)F)c(=O)n3)cc(C)c2O1]

In yet another embodiment the cargo molecule is the Doxorubicin delta-tocotrienlyl carbonate ester, the structure of which is:

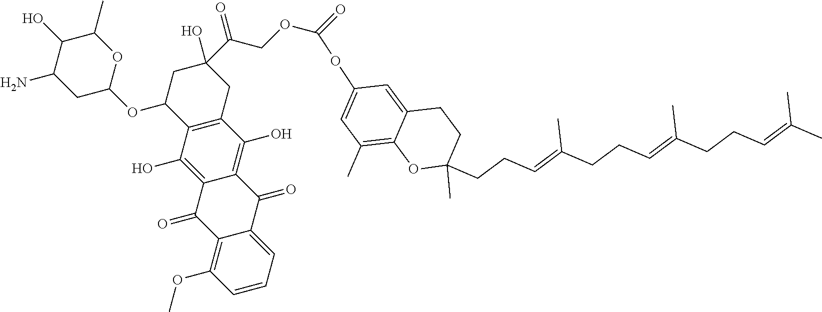 [SMILES: COc1cccc2c1C(=O)c1c(O)c3c(c(O)c1C2=O)CC(O)(C(=O)COC(=O)Oc1cc(C)c2c(c1)CCC(C)(CC/C=C(\C)CC/C=C(\C)CCC=C(C)C)O2)CC3OC1CC(N)C(O)C(C)O1]

Table 3 provides the structure of non-limiting examples of agents (A) useful in the present invention with the hydroxyl or amine group (R) indicated by an arrow.

TABLE 3

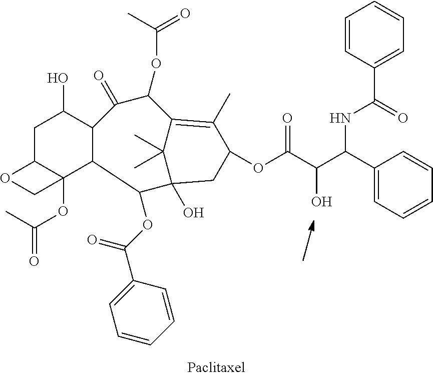 [SMILES: CC(=O)OC1C(=O)C2C(O)CC3OCC3(OC(C)=O)C2C(OC(=O)c2ccccc2)C2(O)CC(OC(=O)C(O)C(NC(=O)c3ccccc3)c3ccccc3)C(C)=C1C2(C)C]

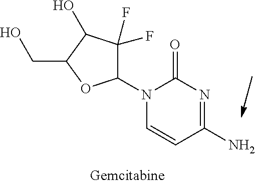 [SMILES: Nc1ccn(C2OC(CO)C(O)C2(F)F)c(=O)n1]

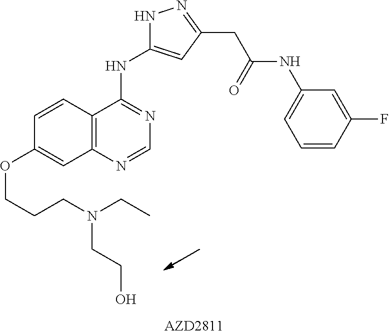 [SMILES: CCN(CCO)CCCOc1ccc2c(Nc3cc(CC(=O)Nc4cccc(F)c4)n[nH]3)ncnc2c1]

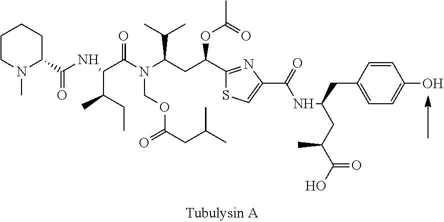 [SMILES: CC[C@@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C]

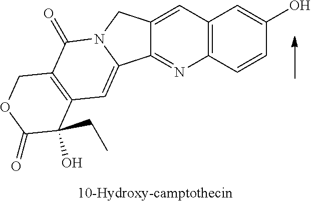 [SMILES: CC[C@@]1(O)C(=O)OCc2c1cc1n(c2=O)Cc2cc3cc(O)ccc3nc2-1]

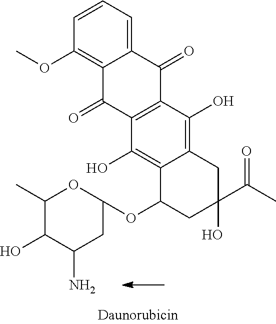 [SMILES: COc1cccc2c1C(=O)c1c(O)c3c(c(O)c1C2=O)CC(O)(C(C)=O)CC3OC1CC(N)C(O)C(C)O1]

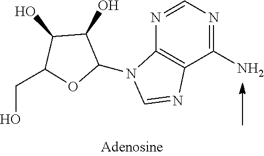 [SMILES: Nc1ncnc2c1ncn2C1OC(CO)[C@@H](O)[C@H]1O]

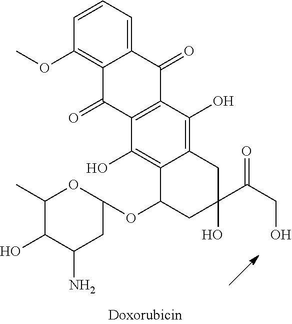 [SMILES: COc1cccc2c1C(=O)c1c(O)c3c(c(O)c1C2=O)CC(O)(C(=O)CO)CC3OC1CC(N)C(O)C(C)O1]

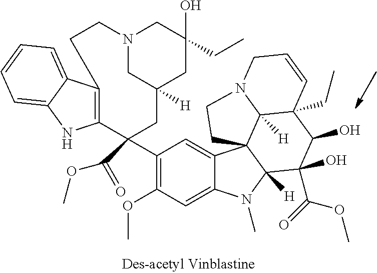

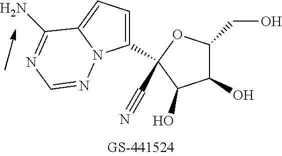 [SMILES: N#C[C@@]1(c2ccc3c(N)ncnn23)O[C@H](CO)[C@@H](O)[C@H]1O]

Table 4 provides non-liming examples of PALM-cargo compositions of formula A-R-L-X.

TABLE 4

| Compound | A | R | L | X |
|---|---|---|---|---|
| 1 | Paclitaxel | OH | Carbonic Acid | γ-Tocotrienol |
| 2 | Paclitaxel | OH | Carbonic Acid | δ-Tocotrienol |
| 3 | Paclitaxel | OH | Carbonic Acid | Cholecalciferol |
| 4 | Paclitaxel | OH | Carbonic Acid | Ergocalciferol |
| 5 | Paclitaxel | OH | succinic acid | Cholesterol |
| 6 | Paclitaxel | OH | succinic acid | γ-Tocotrienol |
| 7 | Paclitaxel | OH | succinic acid | δ-Tocotrienol |
| 8 | Paclitaxel | OH | succinic acid | Cholecalciferol |
| 9 | Paclitaxel | OH | succinic acid | Ergocalciferol |
| 10 | Paclitaxel | OH | Diglycolic Acid | Cholesterol |
| 11 | Paclitaxel | OH | Diglycolic Acid | γ-Tocotrienol |
| 12 | Paclitaxel | OH | Diglycolic Acid | δ-Tocotrienol |
| 13 | Paclitaxel | OH | Diglycolic Acid | Cholecalciferol |
| 14 | Paclitaxel | OH | Diglycolic Acid | Ergocalciferol |
| 15 | Gemcitabine | NH₂ | Carbonic Acid | Cholesterol |
| 16 | Gemcitabine | NH₂ | Carbonic Acid | γ-Tocotrienol |
| 17 | Gemcitabine | NH₂ | Carbonic Acid | δ-Tocotrienol |
| 18 | Gemcitabine | NH₂ | Carbonic Acid | Cholecalciferol |
| 19 | Gemcitabine | NH₂ | Carbonic Acid | Ergocalciferol |
| 20 | Gemcitabine | NH₂ | succinic acid | Cholesterol |
| 21 | Gemcitabine | NH₂ | succinic acid | γ-Tocotrienol |
| 22 | Gemcitabine | NH₂ | succinic acid | δ-Tocotrienol |
| 23 | Gemcitabine | NH₂ | succinic acid | Cholecalciferol |
| 24 | Gemcitabine | NH₂ | succinic acid | Ergocalciferol |
| 25 | Gemcitabine | NH₂ | Diglycolic Acid | Cholesterol |
| 26 | Gemcitabine | NH₂ | Diglycolic Acid | γ-Tocotrienol |
| 27 | Gemcitabine | NH₂ | Diglycolic Acid | δ-Tocotrienol |
| 28 | Gemcitabine | NH₂ | Diglycolic Acid | Cholecalciferol |
| 29 | Gemcitabine | NH₂ | Diglycolic Acid | Ergocalciferol |
| 30 | AZD2811 | OH | Carbonic Acid | Cholesterol |
| 31 | AZD2811 | OH | Carbonic Acid | γ-Tocotrienol |
| 32 | AZD2811 | OH | Carbonic Acid | δ-Tocotrienol |
| 33 | AZD2811 | OH | Carbonic Acid | Cholecalciferol |
| 34 | AZD2811 | OH | Carbonic Acid | Ergocalciferol |
| 35 | AZD2811 | OH | succinic acid | Cholesterol |
| 36 | AZD2811 | OH | succinic acid | γ-Tocotrienol |
| 37 | AZD2811 | OH | succinic acid | δ-Tocotrienol |
| 38 | AZD2811 | OH | succinic acid | Cholecalciferol |
| 39 | AZD2811 | OH | succinic acid | Ergocalciferol |
| 40 | AZD2811 | OH | Diglycolic Acid | Cholesterol |
| 41 | AZD2811 | OH | Diglycolic Acid | γ-Tocotrienol |
| 42 | AZD2811 | OH | Diglycolic Acid | δ-Tocotrienol |
| 43 | AZD2811 | OH | Diglycolic Acid | Cholecalciferol |
| 44 | AZD2811 | OH | Diglycolic Acid | Ergocalciferol |
| 45 | Daunorubicin | NH₂ | Carbonic Acid | Cholesterol |
| 46 | Daunorubicin | NH₂ | succinic acid | γ-Tocotrienol |
| 47 | Daunorubicin | NH₂ | Carbonic Acid | δ-Tocotrienol |
| 48 | Daunorubicin | NH₂ | Carbonic Acid | Cholecalciferol |
| 49 | Daunorubicin | NH₂ | Carbonic Acid | Ergocalciferol |
| 50 | Daunorubicin | NH₂ | succinic acid | Cholesterol |
| 51 | Daunorubicin | NH₂ | succinic acid | γ-Tocotrienol |
| 52 | Daunorubicin | NH₂ | succinic acid | δ-Tocotrienol |
| 53 | Daunorubicin | NH₂ | succinic acid | Cholecalciferol |
| 54 | Daunorubicin | NH₂ | succinic acid | Ergocalciferol |
| 55 | Daunorubicin | NH₂ | Diglycolic Acid | Cholesterol |
| 56 | Daunorubicin | NH₂ | Diglycolic Acid | γ-Tocotrienol |
| 57 | Daunorubicin | NH₂ | Diglycolic Acid | δ-Tocotrienol |
| 58 | Daunorubicin | NH₂ | Diglycolic Acid | Cholecalciferol |
| 59 | Daunorubicin | NH₂ | Diglycolic Acid | Ergocalciferol |
| 60 | 10-Hydroxy- | OH | Carbonic Acid | Cholesterol |
| | camptothecin | | | |
| 61 | 10-Hydroxy- | OH | Carbonic Acid | γ-Tocotrienol |
| | camptothecin | | | |
| 62 | 10-Hydroxy- | OH | Carbonic Acid | δ-Tocotrienol |
| | camptothecin | | | |
| 63 | 10-Hydroxy- | OH | Carbonic Acid | Cholecalciferol |
| | camptothecin | | | |
| 64 | 10-Hydroxy- | OH | Carbonic Acid | Ergocalciferol |
| | camptothecin | | | |
| 65 | 10-Hydroxy- | OH | succinic acid | Cholesterol |
| | camptothecin | | | |
| 66 | 10-Hydroxy- | OH | succinic acid | γ-Tocotrienol |
| | camptothecin | | | |
| 67 | 10-Hydroxy- | OH | succinic acid | δ-Tocotrienol |
| | camptothecin | | | |
| 68 | 10-Hydroxy- | OH | succinic acid | Cholecalciferol |
| | camptothecin | | | |
| 69 | 10-Hydroxy- | OH | succinic acid | Ergocalciferol |
| | camptothecin | | | |
| 70 | 10-Hydroxy- | OH | Diglycolic Acid | Cholesterol |
| | camptothecin | | | |
| 71 | 10-Hydroxy- | OH | Diglycolic Acid | γ-Tocotrienol |
| | camptothecin | | | |
| 72 | 10-Hydroxy- | OH | Diglycolic Acid | δ-Tocotrienol |
| | camptothecin | | | |
| 73 | 10-Hydroxy- | OH | Diglycolic Acid | Cholecalciferol |
| | camptothecin | | | |
| 74 | 10-Hydroxy- | OH | Diglycolic Acid | Ergocalciferol |
| | camptothecin | | | |
| 75 | Adenosine | NH₂ | Carbonic Acid | Cholesterol |
| 76 | Adenosine | NH₂ | Carbonic Acid | γ-Tocotrienol |
| 77 | Adenosine | NH₂ | Carbonic Acid | δ-Tocotrienol |
| 78 | Adenosine | NH₂ | Carbonic Acid | Cholecalciferol |
| 79 | Adenosine | NH₂ | Carbonic Acid | Ergocalciferol |
| 80 | Adenosine | NH₂ | succinic acid | Cholesterol |
| 81 | Adenosine | NH₂ | succinic acid | γ-Tocotrienol |
| 82 | Adenosine | NH₂ | succinic acid | δ-Tocotrienol |
| 83 | Adenosine | NH₂ | succinic acid | Cholecalciferol |
| 84 | Adenosine | NH₂ | succinic acid | Ergocalciferol |
| 85 | Adenosine | NH₂ | Diglycolic Acid | Cholesterol |
| 86 | Adenosine | NH₂ | Diglycolic Acid | γ-Tocotrienol |
| 87 | Adenosine | NH₂ | Diglycolic Acid | δ-Tocotrienol |
| 88 | Adenosine | NH₂ | Diglycolic Acid | Cholecalciferol |
| 89 | Adenosine | NH₂ | Diglycolic Acid | Ergocalciferol |
| 90 | Tubulysin A | OH | Carbonic Acid | Cholesterol |
| 91 | Tubulysin A | OH | Carbonic Acid | γ-Tocotrienol |
| 92 | Tubulysin A | OH | Carbonic Acid | δ-Tocotrienol |
| 93 | Tubulysin A | OH | Carbonic Acid | Cholecalciferol |
| 94 | Tubulysin A | OH | Carbonic Acid | Ergocalciferol |
| 95 | Tubulysin A | OH | succinic acid | Cholesterol |
| 96 | Tubulysin A | OH | succinic acid | γ-Tocotrienol |
| 97 | Tubulysin A | OH | succinic acid | δ-Tocotrienol |
| 98 | Tubulysin A | OH | succinic acid | Cholecalciferol |
| 99 | Tubulysin A | OH | succinic acid | Ergocalciferol |
| 100 | Tubulysin A | OH | Diglycolic Acid | Cholesterol |
| 101 | Tubulysin A | OH | Diglycolic Acid | γ-Tocotrienol |
| 102 | Tubulysin A | OH | Diglycolic Acid | δ-Tocotrienol |
| 103 | Tubulysin A | OH | Diglycolic Acid | Cholecalciferol |
| 104 | Tubulysin A | OH | Diglycolic Acid | Ergocalciferol |
| 105 | Doxorubicin | OH | Carbonic Acid | Cholesterol |
| 106 | Doxorubicin | OH | Carbonic Acid | γ-Tocotrienol |
| 107 | Doxorubicin | OH | Carbonic Acid | δ-Tocotrienol |
| 108 | Doxorubicin | OH | Carbonic Acid | Cholecalciferol |
| 109 | Doxorubicin | OH | Carbonic Acid | Ergocalciferol |
| 110 | Doxorubicin | OH | succinic acid | Cholesterol |
| 111 | Doxorubicin | OH | succinic acid | γ-Tocotrienol |
| 112 | Doxorubicin | OH | succinic acid | δ-Tocotrienol |
| 113 | Doxorubicin | OH | succinic acid | Cholecalciferol |
| 114 | Doxorubicin | OH | succinic acid | Ergocalciferol |
| 115 | Doxorubicin | OH | Diglycolic Acid | Cholesterol |
| 116 | Doxorubicin | OH | Diglycolic Acid | γ-Tocotrienol |
| 117 | Doxorubicin | OH | Diglycolic Acid | δ-Tocotrienol |
| 118 | Doxorubicin | OH | Diglycolic Acid | Cholecalciferol |
| 119 | Doxorubicin | OH | Diglycolic Acid | Ergocalciferol |

So various embodiment of A-R-L-X are comprised of the following: Paclitaxel-OH-carbonic acid-γ-Tocotrienol. Paclitaxel-OH-carbonic acid-δ-tocotrienol. Paclitaxel-OH-carbonic acid-cholecalciferol. Paclitaxel-OH-carbonic acid-ergocalciferol. Paclitaxel-OH-succinic acid-cholesterol. Paclitaxel-OH-succinic acid-γ-tocotrienol. Paclitaxel-OH-succinic acid-δ-tocotrienol. Paclitaxel-OH-succinic acid-cholesterol. Paclitaxel-OH-succinic acid-cholecalciferol. Paclitaxel-OH-succinic acid-ergocalciferol. Paclitaxel-OH-diglycolic acid-γ-tocotrienol. Paclitaxel-OH-diglycolic acid-δ-tocotrienol. Paclitaxel-OH-diglycolic acid-cholecalciferol. Paclitaxel-OH-diglycolic acid-ergocalciferol. Gemcitabine-NH₂-carbonic acid-cholesterol. Gemcitabine-NH₂-carbonic acid-γ-tocotrienol. Gemcitabine-NH₂-carbonic acid-δ-tocotrienol. Gemcitabine-NH₂-carbonic acid-cholecalciferol. Gemcitabine-NH₂-carbonic acid-ergocalciferol. Gemcitabine-NH₂-succinic acid-cholesterol. Gemcitabine-NH₂-succinic acid-γ-tocotrienol. Gemcitabine-NH₂-succinic acid-δ-tocotrienol. Gemcitabine-NH₂-succinic acid-cholecalciferol. Gemcitabine-NH₂-succinic acid-ergocalciferol. Gemcitabine-NH₂-diglycolic acid-cholesterol. Gemcitabine-NH₂-carbonic acid-γ-tocotrienol. Gemcitabine-NH₂-diglycolic acid-δ-tocotrienol. Gemcitabine-NH₂-diglycolic acid-cholecalciferol. Gemcitabine-NH₂-diglycolic acid-ergocalciferol. AZD2811-OH-carbonic acid-γ-tocotrienol. AZD2811-OH-carbonic acid-cholesterol. AZD2811-OH-carbonic acid-γ-tocotrienol. AZD2811-OH-carbonic acid-δ-tocotrienol. AZD2811-OH-carbonic acid-cholecalciferol. AZD2811-OH-carbonic acid-ergocalciferol. AZD2811-OH-succinic acid-cholesterol. AZD2811-OH-succinic acid-γ-tocotrienol. AZD2811-OH-succinic acid-γ-tocotrienol. AZD2811-OH-succinic acid-δ-tocotrienol. AZD2811-OH-succinic acid-cholecalciferol. AZD2811-OH-succinic acid-ergocalciferol. AZD2811-OH-diglycolic acid-γ-tocotrienol. AZD2811-OH-diglycolic acid-cholesterol. AZD2811-OH-diglycolic acid-γ-tocotrienol. AZD2811-OH-diglycolic acid-δ-tocotrienol. AZD2811-OH-diglycolic acid-cholecalciferol. AZD2811-OH-diglycolic acid-ergocalciferol. Daunorubicin-NH₂-carbonic acid-cholesterol. Daunorubicin-NH₂-carbonic acid-γ tocotrienol. Daunorubicin-NH₂-carbonic acid-δ tocotrienol. Daunorubicin-NH₂-carbonic acid-cholecalciferol. Daunorubicin-NH₂-carbonic acid-ergocalciferol. Daunorubicin-NH₂-succinic acid-cholesterol. Daunorubicin-NH₂-succinic acid-γ tocotrienol. Daunorubicin-NH₂-succinic acid-δ tocotrienol. Daunorubicin-NH₂-succinic acid-cholecalciferol. Daunorubicin-NH₂-succinic acid-ergocalciferol. Daunorubicin-NH₂-diglycolic acid-cholesterol. Daunorubicin-NH₂-diglycolic acid-γ-tocotrienol. Daunorubicin-NH₂-diglycolic acid-δ-tocotrienol. Daunorubicin-NH₂-diglycolic acid-cholecalciferol. Daunorubicin-NH₂-diglycolic acid-ergocalciferol. 10-Hydroxy-camptothecin-OH-carbonic acid-cholesterol. 10-Hydroxy-camptothecin-OH-carbonic acid-γ-tocotrienol. 10-Hydroxy-camptothecin-OH-carbonic acid-δ-tocotrienol. 10-Hydroxy-camptothecin-OH-carbonic acid-cholecalciferol. 10-Hydroxy-camptothecin-OH-carbonic acid-ergocalciferol. 10-Hydroxy-camptothecin-OH-succinic acid-γ-tocotrienol. 10-Hydroxy-camptothecin-OH-succinic acid-cholesterol. 10-Hydroxy-camptothecin-OH-succinic acid-γ-tocotrienol. 10-Hydroxy-camptothecin-OH-succinic acid-δ-tocotrienol. 10-Hydroxy-camptothecin-OH-succinic acid-cholecalciferol. 10-Hydroxy-camptothecin-OH-succinic acid-ergocalciferol. 10-Hydroxy-camptothecin-OH-diglycolic acid-γ-tocotrienol. 10-Hydroxy-camptothecin-OH-diglycolic acid-cholesterol. 10-Hydroxy-camptothecin-OH-diglycolic acid-γ-tocotrienol. 10-Hydroxy-camptothecin-OH-diglycolic acid-δ-tocotrienol. 10-Hydroxy-camptothecin-OH-diglycolic acid-cholecalciferol. 10-Hydroxy-camptothecin-OH-diglycolic acid-ergocalciferol. AdenosineAdenosine-NH₂-carbonic acid-cholesterol. Adenosine-NH₂-carbonic acid-γ tocotrienol. Adenosine-NH₂-carbonic acid-δ tocotrienol. Adenosine-NH₂-carbonic acid-cholecalciferol. Adenosine-NH₂-carbonic acid-ergocalciferol. Adenosine-NH₂-succinic acid-cholesterol. Adenosine-NH₂-succinic acid-γ tocotrienol. Adenosine-NH₂-succinic acid-δ tocotrienol. Adenosine-NH₂-succinic acid-cholecalciferol. Adenosine-NH₂-succinic acid-ergocalciferol. Adenosine-NH₂-diglycolic acid-cholesterol. Adenosine-NH₂-diglycolic acid-γ-tocotrienol. Adenosine-NH₂-diglycolic acid-δ-tocotrienol. Adenosine-NH₂-diglycolic acid-cholecalciferol. Adenosine-NH₂-diglycolic acid-ergocalciferol. Tubulysin A-OH-carbonic acid-γ-tocotrienol. Tubulysin A-OH-carbonic acid-cholesterol. Tubulysin A-OH-carbonic acid-γ-tocotrienol. Tubulysin A-OH-carbonic acid-δ-tocotrienol. Tubulysin A-OH-carbonic acid-cholecalciferol. Tubulysin A-OH-carbonic acid-ergocalciferol. Tubulysin A-OH-succinic acid-cholesterol. Tubulysin A-OH-succinic acid-γ-tocotrienol. Tubulysin A-OH-succinic acid-γ-tocotrienol. Tubulysin A-OH-succinic acid-δ-tocotrienol. Tubulysin A-OH-succinic acid-cholecalciferol. Tubulysin A-OH-succinic acid-ergocalciferol. Tubulysin A-OH-diglycolic acid-γ-tocotrienol. Tubulysin A-OH-diglycolic acid-cholesterol. Tubulysin A-OH-diglycolic acid-γ-tocotrienol. Tubulysin A-OH-diglycolic acid-δ-tocotrienol. Tubulysin A-OH-diglycolic acid-cholecalciferol. Tubulysin A-OH-diglycolic acid-ergocalciferol. Doxorubicin-OH-carbonic acid-γ-tocotrienol. Doxorubicin-OH-carbonic acid-cholesterol. Doxorubicin-OH-carbonic acid-γ-tocotrienol. Doxorubicin-OH-carbonic acid-δ-tocotrienol. Doxorubicin-OH-carbonic acid-cholecalciferol. Doxorubicin-OH-carbonic acid-ergocalciferol. Doxorubicin-OH-succinic acid-cholesterol. Doxorubicin-OH-succinic acid-γ-tocotrienol. Doxorubicin-OH-succinic acid-γ-tocotrienol. Doxorubicin-OH-succinic acid-δ-tocotrienol. Doxorubicin-OH-succinic acid-cholecalciferol. Doxorubicin-OH-succinic acid-ergocalciferol. Doxorubicin-OH-diglycolic acid-γ-tocotrienol. Doxorubicin-OH-diglycolic acid-cholesterol. Doxorubicin-OH-diglycolic acid-γ-tocotrienol. Doxorubicin-OH-diglycolic acid-δ-tocotrienol. Doxorubicin-OH-diglycolic acid-cholecalciferol. Doxorubicin-OH-diglycolic acid-ergocalciferol.

A fourth aspect of the disclosure provides for a surprisingly effective co-lyophilization techniques to produce PALM or PALM-cargo molecule compositions from a homogenous solvent phase composed of tert-butyl alcohol and water. The advantages of this approach are: 1) all PALM constituents including peptide, phospholipid and optional lipophilic cargo (e.g. paclitaxel-2′-cholesteryl carbonate), are co-solubilized in a single solvent phase, 2) the solvent components are totally miscible and well-suited to removal by standard lyophilization procedure, 3) the procedures avoids potentially toxic substances because tert-butyl alcohol is a low toxicity, class 3 solvent and 4) the resultant dried lyophilizate enables opportunities for greater stability during storage than is possible with aqueous preparations.

The solvent mixture used to prepare PALM is preferably a mixture of tert-butyl alcohol (TBA) and water. In one embodiment the percent ration of TBA to water is between about 70%:30% to about 90%:10%. In another embodiment the ratio is between about 75%:25% and about 85%:15%. In yet another embodiment the ratio is 80%:20%.

One embodiment of the fourth aspect provides a process for preparing PALM comprises the steps:
i) solubilizing an amphiphilic peptide in a first solvent mixture to provide a peptide solution;
ii) solubilizing a sphingomyelin in a second solvent mixture to provide a sphingomyelin solution
iii) solubilizing an additional phospholipid in a third solvent mixture to provide a phospholipid solution;
iv) combining the peptide solution, the sphingomyelin solution and the phospholipid solution to form a peptide/sphingomyelin/phospholipid solution; and
v) lyophilizing the peptide/sphingomyelin/phospholipid solution, wherein steps i), ii), and iii) are performed in any order; and wherein the first, second, and third solvent mixture comprises tert-butyl alcohol and water.

Another embodiment of the fourth aspect of the disclosure provides a process for preparing PALM comprises the steps:
i) combining an amphiphilic peptide, sphingomyelin and an additional phospholipid, to form a peptide/sphingomyelin/phospholipid mixture;
ii) solubilizing the peptide/sphingomyelin/phospholipid mixture in a solvent mixture to form a peptide sphingomyelin/phospholipid solution; and
iii) lyophilizing the peptide/phospholipid solution, wherein the solvent mixture comprises tert-butyl alcohol and water.

The fourth aspect of the present disclosure additionally provides a process for preparing PALM comprising a cargo molecule to form a PALM-cargo molecule complex. To prepare a PALM-cargo molecule complex, the peptide, sphingomyelin, one or more additional phospholipids and a cargo molecule are each separately prepared in a solvent mixture and, depending on the desired formulation, are combined in specific molar ratios. Alternately, the peptide, sphingomyelin, one or more additional phospholipid and a cargo molecule can be combined directly, without prior solubilization, and then brought into solution with the desired solvent mixture prior to lyophilization.

One embodiment of the fourth aspect of the disclosure provides a process for preparing a PALM-cargo molecule complex comprising the steps:
i) solubilizing an amphiphilic peptide in a first solvent mixture to provide a peptide solution;
ii) solubilizing a sphingomyelin in a second solvent mixture to provide a sphingomyelin solution;
iii) solubilizing an additional phospholipid in a third solvent mixture to provide a phospholipid solution;
iv) solubilizing a cargo molecule in a fourth solvent mixture to provide a cargo molecule solution;
v) combining the peptide solution, the sphingomyelin solution, the phospholipid solution, and the cargo molecule solution to form a peptide/sphingomyelin/phospholipid/cargo molecule solution; and
vi) lyophilizing the peptide/sphingomyelin/phospholipid/cargo molecule solution, wherein steps i) ii), iii) and iv) are performed in any order; and wherein the first, second, third and fourth solvent mixture comprise tert-butyl alcohol and water.

Another embodiment of preparing a PALM-cargo molecule complex comprises the steps:
i) combining an amphiphilic peptide, sphingomyelin, an additional phospholipid and a cargo molecule, to form a peptide/sphingomyelin/phospholipid/cargo molecule mixture;
ii) solubilizing the peptide/sphingomyelin/phospholipid/cargo molecule mixture in a solvent mixture to form a peptide/phospholipid solution; and
iii) lyophilizing the peptide/sphingomyelin/phospholipid/cargo molecule solution, wherein the solvent mixture comprises tert-butyl alcohol and water

The resultant lyophilized cake can be stored for long periods of time and will remain stable. The lyophilized product is rehydrated by adding any suitable aqueous solution, e.g., water or saline, followed by gentle swirling of the contents. Reconstitution of PALM lyophilizates can be enhanced by incubation of the PALM solution at 50° C. for from 5 to 30 minutes. The solution is then filter sterilized (0.2 μm) and stored at 4° C. Alternately, the solvent mixture comprising the peptide, phospholipid and the cargo molecule is filter sterilized prior to lyophilization.

A fifth aspect of the present disclosure provides methods for treating a disorder comprising administering to a subject in need thereof, an effective amount of a PALM-cargo composition according to any one the embodiments of the third aspect of the disclosure.

Scavenger receptor BI (SR-BI) is a membrane receptor that binds apolipoprotein A-I, the principal protein component of HDL, to facilitate cellular transport of cholesterol. Cholesterol is an essential nutrient for proliferating cells like those found in malignant tumors. SR-BI is highly expressed in many tumor cells, including but not limited to breast, prostate, colorectal, pancreatic, adrenal, skin, nasopharyngeal and ovarian cancers. Some amphiphilic peptides are also recognized and bound by SR-BI. PALM are formed from combinations of phospholipid and amphiphilic peptides designed to bind to SR-BI and thereby to selectively deliver cargo molecules to SR-BI-positive cells.

Accordingly, one embodiment of the fifth aspect of the present disclosure provides for methods of treating disorders associated with the overexpression of SR-BI receptors comprising administering a PALM-drug to a subject in need thereof. In one embodiment the method is a method of treating cancer by administering a PALM-cargo molecule composition to a subject in need thereof.

For pharmaceutical use, lyophilized PALM may be provided in single dose or multiple dose containers that can be conveniently reconstituted at the point of use, e.g., hospital or doctor's office using standard diluents such as sterile water for injection, normal sterile saline or sterile 5% dextrose solution. Suitable containers are then aseptically filled with the sterilized mixture, lyophilized and sealed appropriately to maintain sterility of the lyophilized material. Suitable containers include but are not limited to a vial comprising a rubber seal, or the equivalent, that allows for introduction of a diluent for reconstitution, e.g., via a syringe. Such PALM preparations are suitable for parenteral administration including intravenous, subcutaneous, intramuscular, intraperitoneal injection.

For pharmaceutical use, lyophilized PALM may be provided in single dose or multiple dose containers that can be conveniently reconstituted at the point of use, e.g., hospital or doctor's office using standard diluents such as sterile water for injection, normal sterile saline or sterile 5% dextrose solution. Suitable containers are then aseptically filled with the sterilized mixture, lyophilized and sealed appropriately to maintain sterility of the lyophilized material. Suitable containers include but are not limited to a vial comprising a rubber seal, or the equivalent, that allows for introduction of a diluent for reconstitution, e.g., via a syringe. Such PALM preparations are suitable for parenteral administration including intravenous, subcutaneous, intramuscular, intraperitoneal injection.

Total daily dose of the PALM of the invention to be administered to a human or other mammal host in single or divided doses may be in amounts, for example, from 0.1 to 300 mg/kg body weight daily and more usually 0.1 to 200 mg/kg body weight daily, or the dose, from 0.1 to 100 mg/kg body weight daily.

In one embodiment of the invention, the dose of the PALM-chemotherapeutic agent nanoparticles (in total), is in the range of 0.1 to 300 mg/kg, the range of 5 to 200 mg/kg, the range of 10 to 100 mg/kg, or the range of 10 mg/kg to 50 mg/kg. In a further embodiment of the invention, the dose of PALM molecules and the chemotherapeutic agent (in total), is about 5 mg/kg, 10 mg/kg, 20 mg/kg, 40 mg/kg, 50 mg/kg, 60 mg/kg, 70 mg/kg, 80 mg/kg, 90 mg/kg, or 100 mg/kg, 200 mg/kg, 300 mg/kg. The dose can be administered once a day. The dose can be administered three times a week. Alternatively, the dose can be administered twice a week. Alternatively, the dose can be administered once a week. In another embodiment the dose can be administered once a month.

In related embodiments, the amount of chemotherapeutic agent in the combination of PALM and chemotherapeutic agent, the dose may range from about 0.01 mg to about 35 mg per kilogram body weight, about 0.01 mg to about 30 mg per kilogram body weight, about 0.01 mg to about 25 mg per kilogram body weight, about 0.01 to about 20 mg per kilogram body weight, or about 0.01 to about 10 mg per kilogram body weight, of the patient. In further related embodiments, the amount of chemotherapeutic agent in the composition with the PALM molecules is a prescribed Food and Drug Administration (FDA USA) or European Medicines Agency (EMA) approved dose of chemotherapeutic for the treatment of cancer the patient may have or is treated with.

In one embodiment of the invention, the cancer being treated is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In yet another embodiment of the invention, the cancer being treated is ovarian cancer, cervical cancer, colorectal cancer, prostate cancer, breast cancer, gastric adenocarcinoma, head and neck cancer, testicular cancer, leukemia, neuroblastoma, Hodgkin's lymphoma, non-Hodgkin's lymphoma, and non-small cell lung cancer.

In one embodiments of the invention, the cancer is treated with a cargo molecule that is paclitaxel 2′-cholesteryl carbonate. In another embodiment the cancer is treated with a cargo molecule that is paclitaxel 2′-δ-tocotrienyl carbonate. In yet another embodiment the cancer is treated with a cargo molecule that is docetaxel 2′-cholesteryl carbonate. In another embodiment the cancer is treated with a cargo molecule that is the cholesteryl carbonate ester of 10-hydroxycamptothecin. In yet another embodiment the cancer is treated with a cargo molecule that is cholesteryl carbonate ester of 7-ethyl-10-hydroxycamptothecin. In another embodiment, the cancer is treated with a cargo molecule that is the cholesteryl carbonate ester of sirolimus. In another embodiment, the cancer is treated with a cargo molecule that is the cholesteryl carbonate ester of gemcitabine. In yet another embodiment, the cancer is treated with a cargo molecule that is the cholesteryl carbonate ester of tubulysin A. In another embodiment, the cancer is treated with a cargo molecule that is a cholesteryl carbonate ester of morphine. In another embodiment, the cancer is treated with a cargo molecule that is a cholesteryl carbonate ester of hydromorphone. In yet another embodiment, the cancer is treated with a cargo molecule that is a cholesteryl carbonate ester of codeine.

In another embodiment of the invention, the cancer is treated with a cargo molecule that is the cholesteryl carbonate ester of gemcitabine (Cholesteryl (N⁴)-Gemcitabine Carbamate). In another embodiment of the invention, the cancer is treated with a cargo molecule that is the cholesteryl carbonate ester of doxorubicin. In another embodiment of the invention, the cancer is treated with a cargo molecule that is the cholesteryl carbonate ester of adenosine. Or the cancer is treated with the cargo molecule is the cholesteryl carbonate ester of vincristine. In yet another embodiment the cancer is treated with the cargo molecule that is delta-tocotrienyl carbonate ester of paclitaxel. In still another embodiment the cancer is treated with the cargo molecule that is the gemcitabine delta-tocotrienyl carbamate ester. In yet another embodiment the cancer is treated with the cargo molecule that is the Doxorubicin delta-tocotrienyl carbonate ester.

All sequences used in the FIGs. and Examples are included in Table 5.

TABLE 5

| SEQ ID NO: | Peptide Sequence |
|---|---|
| 1 | DVFQKL{Aib}ELFNQLLEKWKQV |
| 2 | DVFQKLVELFNQLLEKWKQV |
| 3 | DV{Aib}QKLFELFNQLLEKWKQV |
| 4 | DV{Aib}QKFLELFNQLLEKWKQV |
| 5 | DVFQKL{Aib}ELFNQL{Aib}EKWKQV |
| 6 | DVFQKL{Aib}ELFNQL{Aib}EKFKQV |
| 7 | D{Aib}FQKL{Aib}ELFNQL{Aib}EKWKQV |
| 8 | DVFQKL{Aib}ELFKELLEKWKQV |
| 9 | DVFQKL{Aib}ELFQQL{Aib}EKWKQV |
| 10 | DVFQKL{Aib}ELFQNL{Aib}EKWKQV |
| 11ª | PVLDLFRELLNELLEALKQKLK |

^{a}LAP642 peptide, Homan et al. 2013

### EXAMPLES

### Example 1 Peptide Synthesis and Purification

Peptides were produced by standard Fmoc solid-phase synthesis techniques at GenScript USA, Inc. (Piscataway, N.J.). The counter ion for all peptides was acetate. Certain peptides were modified at the terminal amino acids by acetylation of the N-terminus and amidation of the C-terminus by standard procedures. Peptides were chromatographically purified to greater than 95% purity by a standard high-performance liquid chromatography method for peptide purification. Purity was confirmed by HPLC and mass spectroscopic analysis.

### Example 2 Paclitaxel 2′-Cholesteryl Carbonate (XC) Synthesis (1)

Fifty milligrams of paclitaxel was dissolved in 2 ml of chloroform and then combined with 1.5 molar excess of cholesterol chloroformate in 2 ml of chloroform plus 4 ml of N,N-diisopropylethylamine and 2 ml acetonitrile. The mixture was stirred overnight at ambient temperature and then dried on a rotary evaporator. The resulting off-white precipitate was dissolved in ethyl acetate/hexane (3:1) and extracted with water, dried, and then dissolved in chloroform. The formation of the product was confirmed by thin-layer chromatography using ethyl acetate/hexane (3:1) as the mobile phase (Rf of paclitaxel 0.4, Rf of Tax-Choi 0.92). Further purification of the product was then carried out on a silica gel column using ethyl acetate/hexane (3:1) as the mobile phase to yield the titled compound (1). The structure was confirmed by mass spectrometry and NMR analysis.

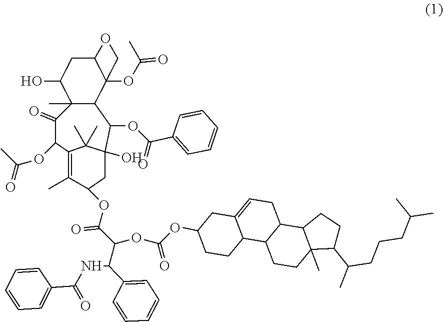 [SMILES: CC(=O)OC1C(=O)C2(C)C(O)CC3OCC3(OC(C)=O)C2C(OC(=O)c2ccccc2)C2(O)CC(OC(=O)C(OC(=O)OC3CCC4C(=CCC5C4CCC4(C)C(C(C)CCCC(C)C)CCC54)C3)C(NC(=O)c3ccccc3)c3ccccc3)C(C)=C1C2(C)C]

### Example 3 Synthesis of Paclitaxel 2′-Palmitate (C₁₆PTX) (2)

One mole equivalent of paclitaxel (50 mg) was combined with 1.2 mole equivalents of palmitoyl chloride (19.3 mg) in 1 ml dry chloroform on ice. Ten microliters triethylamine was added. The mixture was gently stirred and held on ice for 15 min, followed by equilibration to room temperature for 4 hours. Chloroform (4 ml) was added followed by 5 ml of 10% sodium bicarbonate. The organic layer was isolated. The aqueous phase was extracted with 2 successive 5 ml volumes of chloroform. The combined organic phase was dried by rotary evaporation. The dried residue was dissolved in ethyl acetate/hexane (3:1) and chromatographed on a silica gel column, as in Example 1. The product identity was confirmed by mass spectroscopy and NMR.

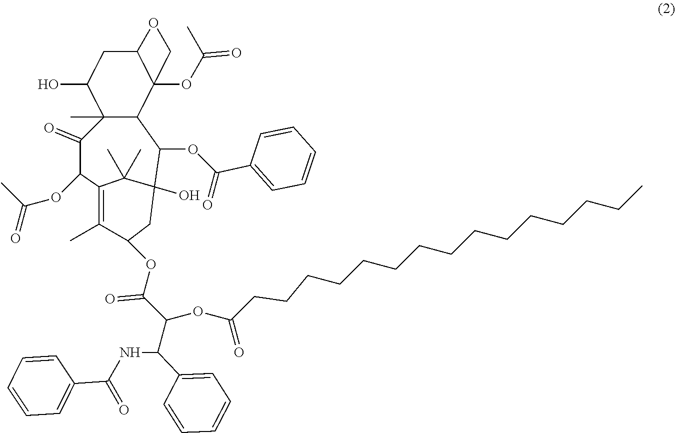 [SMILES: CCCCCCCCCCCCCCCC(=O)OC(C(=O)OC1CC2(O)C(OC(=O)c3ccccc3)C3C4(OC(C)=O)COC4CC(O)C3(C)C(=O)C(OC(C)=O)C(=C1C)C2(C)C)C(NC(=O)c1ccccc1)c1ccccc1]

### Example 4 Paclitaxel 2′-δ-Tocotrienyl Carbonate (XT3) (4)

Step 1. Synthesis of the p-Nitrophenyl Carbonate of Delta-Tocotrienol

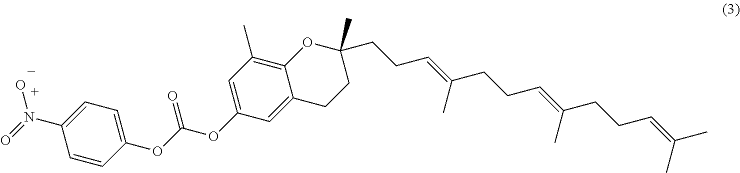

4-Nitrophenyl chloroformate (51 Mg, 0.25 mmol) and triethylmine (35 μL, 0.25 mmol) were added to a solution of delta-tocotrienol (25 Mg, 0.0629 mmol) in anhydrous methylene chloride (1.5 mL) at room temperature. The reaction mixture was stirred for 24 h and then concentrated by rotary evaporation. The desired product (3) was obtained by preparative TLC using ethyl acetate/heptanes (10:90) as eluent. The product was a yellow powder (18 Mg). ¹H NMR (CDCl₃): δ 8.30 (d, 2H), 7.45 (d, 2H), 6.80 (dd, 2H), 5.05-5.20 (m, 3H), 2.72-2.78 (t, 2H), 2.18 (s, 4H), 1.95-2.15 (m, 4H), 1.72-1.85 (m, 4H), 1.68 (s, 3H), 1.55-1.62 (br s, 12H), 1.30 (br s, 5H).

Step 2. Synthesis of Delta-Tocotrienol Carbonate of Paclitaxel (4)

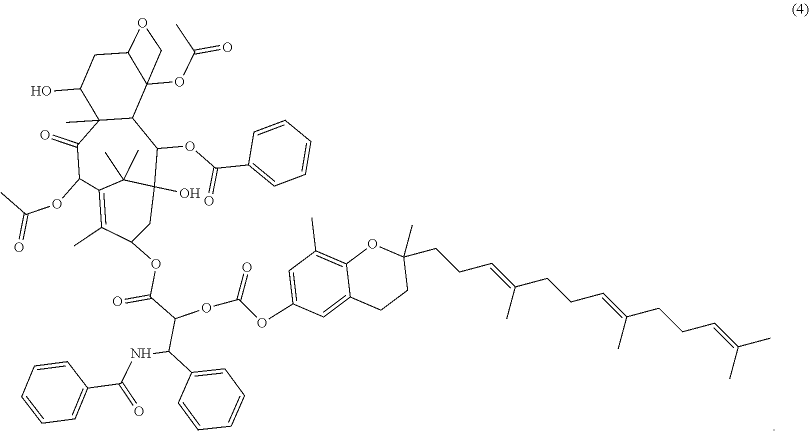 [SMILES: CC(=O)OC1C(=O)C2(C)C(O)CC3OCC3(OC(C)=O)C2C(OC(=O)c2ccccc2)C2(O)CC(OC(=O)C(OC(=O)Oc3cc(C)c4c(c3)CCC(C)(CC/C=C(\C)CC/C=C(\C)CCC=C(C)C)O4)C(NC(=O)c3ccccc3)c3ccccc3)C(C)=C1C2(C)C]

A solution of compound (3) (18 mg, step 1 product) in methylene chloride (2 mL), paclitaxel (28 mg) and DMAP (10 Mg) were combined at room temperature. The mixture was stirred at room temperature for 24 h. The mixture was concentrated and purified by preparative TLC using ethyl acetate/heptanes (50:50) as eluent. The desired product (4) (17 Mg) was obtained as a colorless solid. TLC Analysis (Rf 0.25, EA/Hexanes: 1:1). 1H NMR (CDCl₃): δ 8.20 (d, 2H), 7.75 (d, 2H), 7.60-7.62 (m, 1H), 7.30-7.52 (m, 9H), 6.90-6.95 (d, 1H), 6.60-6.75 (dd, 2H), 6.20-6.30 (m, 2H), 6.00-6.05 (m, 1H), 5.70-5.75 (d, 1H), 5.50 (s, 1H), 5.10-5.20 (br s, 2H), 4.95-5.00 (d, 1H), 4.30-4.35 (br s, 1H), 4.20-4.30 (dd, 2H), 3.75-3.80 (d, 1H), 2.70-2.75 (m, 2H), 2.30-2.60 (m, 7H), 2.23-2.27 (m, 11H), 1.50-2.20 (m, 26H), 1.25 (m, 9H), 1.15 (s, 3H)

Example 5 Peptide Amphiphile Lipid Micelle (PALM) Preparation Separate stock solutions of peptide and phospholipids were prepared in a solvent mixture composed of 80% tert-butyl alcohol (TBA) and 20% water to obtain separate solutions of 10 mM peptide, 20 mM 1-palmitoyl-2-oleoyl-phosphatidylcholine (POPC) and 20 mM egg SM. Aliquots of stock solution were combined to obtain a final solution containing 10 mole equivalents of peptide, 42 mole equivalents of phosphatidylcholine and 18 mole equivalents of SM. The solutions were combined, rapidly frozen (−70° C.), and lyophilized at −20° C. for 24 hours followed by 12-16 hours of additional lyophilization. The resultant lyophilized cakes were rehydrated by addition of Dulbecco's phosphate buffered saline followed by gentle swirling of the contents. Formation of PALM was completed by incubating the PALM solution at 50° C. for 10 minutes. Some peptide complexes remained turbid upon heating and were also subjected to one cycle of freezing to −80° C. followed by thawing to room temperature in an attempt to obtain a clear solution. The qualities of the PALM preparations were evident in their appearance. A visually clear preparation indicated any nanoparticles that had formed were less than approximately 40 nm diameter, the lower limit of the Tyndall effect.

### Example 6 Preparation of PALM Containing the Fluorescent Dye DiI

The procedure essentially followed that described in Example 5. A 40 μl aliquot of 10 mM or peptide was combined with 56 μl of 20 mM POPC, 24 μl 20 mM SM(egg) and 16 μl 2.5 mM DiI in a small glass vial. The peptide and lipid stock solutions were prepared in 80% TBA/20% water. The DiI stock was prepared in 92% TBA/8% water. The solution was lyophilized, and the resultant cake was rehydrated by addition of 0.2 ml of Dulbeco's phosphate buffered saline. The solution was briefly swirled, water bath sonicated (for approx. 15 sec.) and placed in a 50° C. heating block for 20 minutes.

### Example 7 Determination of PALM Size

The size and size uniformity of the PALM preparations was determined by DLS and SEC. Sizes based on hydrodynamic mean diameters were determined by DLS with a Nicomp 370 particle size analyzer. The analyzer was calibrated with latex standards. Particle sizes referred to herein and in the claims are calculated by DLS as described above unless clearly indicated otherwise.

The relative hydrodynamic size of PALM particles was also determined by SEC with a GE Superose 6 Increase column, (10×300 mm) connected to a Beckman/Coulter Model 126 pump and a Model 128 diode array detector. The mobile phase (150 mM NaCl, 6 mM NaPO4 (pH 7.4)) flow rate was 0.5 ml/min. The eluent was monitored at 215 and 280 nm wavelengths. System performance was confirmed by injection of protein molecular weight standards (FIG. 2).

### Example 8 Preparation of PALM Containing Miriplatin

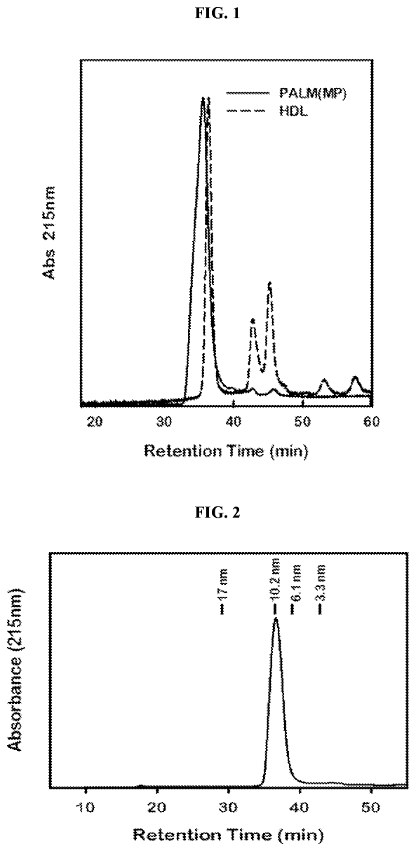

A 50 μl aliquot of 10 mM SEQ ID NO:1 in 80% TBA/20% water corresponding to 2.5 mole equivalents of peptide was combined with 3 mole equivalents of POPC and 7 mole equivalents of egg SM from 40 mM and 20 mM stock solutions, respectively, made up of the same solvent mixture. To this was added 0.75 mole equivalents of miriplatin (MedKoo Biosciences, Raleigh, N.C.) from a 1 mM stock solution prepared with 100% TBA. The solution was lyophilized and the resultant cake was rehydrated by addition of 0.4 ml of 5% dextrose in water. The solution was briefly swirled, water bath sonicated (for approx. 15 sec.) and placed in a 50° C. heating block for 20 minutes. The resultant clear solution was passed through a 0.2 μm pore size, polyethersulfone, sterilization filter and stored at 4° C. Particle size analysis (Example 7) by DLS indicated a hydrodynamic mean diameter of 8 nm. SEC confirmed a single particle population comparable to HDL in size. The SEC chromatogram is shown in FIG. 1 (miriplatin (solid line), human HDL (dashed line)).

### Example 9 Preparation of PALM Containing Paclitaxel 2′-Cholesteryl Carbonate (XC)

The preparation of PALM containing XC was essentially as described in Example 6 with the following exceptions. The peptide was SEQ ID NO:1. A total of 20 microliters of XC from a 10 mM stock solution in 92% TBA/8% water was combined with the other PALM components, in place of DiI. The solution was lyophilized and the resultant cake was rehydrated with 0.4 ml of Dulbecco's phosphate buffered saline. The hydrodynamic mean diameter of this preparation, determined by DLS, was 9 nm (Example 7). Size analysis by SEC indicated a single particle population principally 10 nm in diameter (FIG. 2).

### Example 10 Preparation of PALM Containing Paclitaxel 2′-δ-Tocotrienyl Carbonate (XT3)

A 50 μl aliquot of 10 mM of the peptide of SEQ ID NO:1 in 80% TBA/20% water corresponding to 2.5 mole equivalents of peptide was combined with 7 mole equivalents of POPC and 3 mole equivalents of egg SM from 20 mM stock solutions, made up of the same solvent mixture. To this was added 1.25 mole equivalents of XT3 from a 10 mM stock solution in 92% TBA/8% water. The lyophilized cake was rehydrated with 0.4 ml of Dulbeco's phosphate buffered saline.

### Example 11 R4F is Unsuitable for Preparation of PALM Containing 2′-Paclitaxel δ-Tocotrienyl Carbonate (XT3)

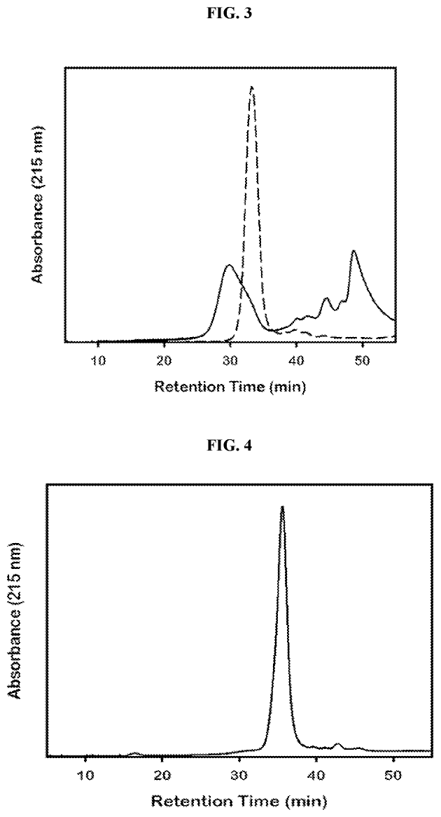

A PALM preparation was conducted as in Example 9 with SEQ ID NO:1 and a second PALM preparation was made by the same method with R4F peptide (Table 1). Unlike PALM made with the peptide of SEQ ID NO:1, which remained a clear solution at room temperature and 4° C., PALM containing R4F was a clear solution at room temperature but became a hazy gel at 4° C. The gel returned to clear liquid upon warming to room temperature. The PALM preparations were analyzed for size (Example 7). Dynamic light scattering indicated the PALM with the peptide of SEQ ID NO:1 had a mean hydrodynamic diameter of 8 nm (volume intensity). The same analysis for PALM with R4F showed 94% of particle population at a mean hydrodynamic diameter of 11 nm with the remainder at 32 nm. SEC confirmed the uniform size distribution of the PALM with the peptide of SEQ ID NO:1 (FIG. 3). In contrast, the PALM with R4F showed a range of peaks eluting at sizes larger than the SEQ ID NO:1 PALM to sizes smaller than 8 nm. These results indicate R4F is not a suitable peptide for PALM preparation.

### Example 12 Fenretinide is Loaded in PALM Prepared with the Peptide of SEQ ID NO:1

A 35 μl aliquot of 10 mM the peptide of SEQ ID NO:1 in 80% TBA/20% water corresponding to 2.5 mole equivalents of peptide was combined with 3 mole equivalents of POPC and 7 mole equivalents of egg SM from 40 mM and 20 mM stock solutions, respectively, made up in the same solvent mixture. Two mole equivalents of 20 mM fenretinide, in the same solvent mixture, were also added. The solution was lyophilized and the resultant cake was rehydrated with 0.325 ml phosphate buffered saline. The solution became clear within 20 min at 50° C. Analysis by SEC (Example 7) indicated all components eluted as a single peak in the 8 nm-10 nm diameter range (FIG. 4).

### Example 13 Quantification of Paclitaxel

Paclitaxel, XT3 and XC are extracted from aqueous samples by mixing 1 volume aqueous sample with 4 volumes of ethyl acetate/acetone/methanol (70/30/5 v/v). The upper organic layer, obtained after shaking and centrifugation, is collected, dried by solvent evaporation and vacuum, and re-dissolved in HPLC mobile phase (methanol/water (65/35 v/v)). A 20 μL aliquot of reconstituted sample is injected on an HPLC at a flow rate of 1.2 ml/minute through a Macherey-Nagel column (4×250 mm with Nucleosil 10-5 C18) and detected with a UV detector at 230 nm wavelength.

### Example 14 PALM Containing Miriplatin Inhibits PC-3 Cell Growth as Well as Cisplatin

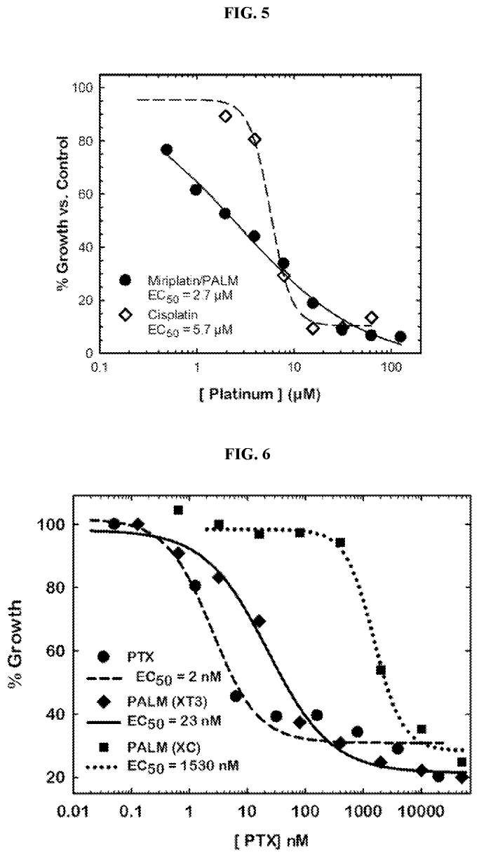

PC-3 cells (American Type Culture Collection, CRL-1435) were seeded in 96-well plates at a density of 5×10³ cells per well (100 μL) and grown till approximately 70% confluence (24 hour) in F-12K medium supplemented with 10% fetal bovine serum. Next, growth medium was replaced by either 100 μL fresh growth medium (control) or by growth medium supplemented with various concentrations of cisplatin (e.g. 0 μM and 0.1 to 100 μM final concentration in medium) added from 100-fold concentrated stock solutions prepared in 5% dextrose or with equivalent amounts of MP in PALM, as prepared in Example 8. Each condition was tested in triplicate. Plates were incubated for 48 hours. Cell viability was assayed by adding 20 μl of 5 mg/ml MTT in Dulbecco's phosphate buffered saline (with calcium and magnesium) and incubating for 3 hours. Next, media were carefully removed and replaced by 200 μL of dimethylsulfoxide (DMSO). The plates were agitated gently for 15 minutes on an orbital shaker. The absorbance of each well was read at 570 nm. The concentration resulting in 50% growth inhibition (IC50) was determined by non-linear regression fit of the data to the logistic equation. The average absorbance of the control wells represented 100% growth (FIG. 5).

### Example 15 XT3 in PALM is More Active than XC in PALM in Blocking SKOV-3 Cell Growth XC

SKOV-3 ovarian cancer cells (American Type Culture Collection, HTB-77) were seeded in 96-well plates at a density of 5×10³ cells per well (100 μL) and grown till approximately 70% confluence in McCoy's medium supplemented with 10% fetal bovine serum. Next, growth medium was replaced by either 100 μL fresh growth medium (control) or by growth medium supplemented with various concentrations of paclitaxel, PALM(XC) or PALM(XT3). A test solution of 20 μM paclitaxel was prepared by dilution of a 5 mM stock solution of paclitaxel in DMSO into growth medium followed by filter sterilization (0.2 μm filter). An aliquot of the 20 μM solution was diluted 5-fold in growth medium to obtain 404 paclitaxel. The 5-fold dilution process was continued with the 404 to obtain an 800 nM paclitaxel solution. This process was continued until a concentration of 0.051 nM paclitaxel in growth medium had been obtained. Four 1004 aliquots of each of the 9 solutions thus obtained were applied to separate wells containing cells. A similar but modified process was used for preparation of PALM(XC) and PALM(XT3) test solutions. The highest concentration tested was 5004, which was prepared by dilution of 1 mM preparations (Examples 9, 10) of PALM(XC) and PALM(XT3) in growth medium followed by filter sterilization. The lowest concentration obtained in the process of 5-fold dilution of each newest dilution repeated 8 times was 0.13 nM. Cells were incubated with the test solutions for 72 hours. At the end of this period cell viability was determined by MTT assay, as in Example 14. (FIG. 6).

### Example 16 SR-BI Selectivity of PALM in BHK(SR-BI) Cells

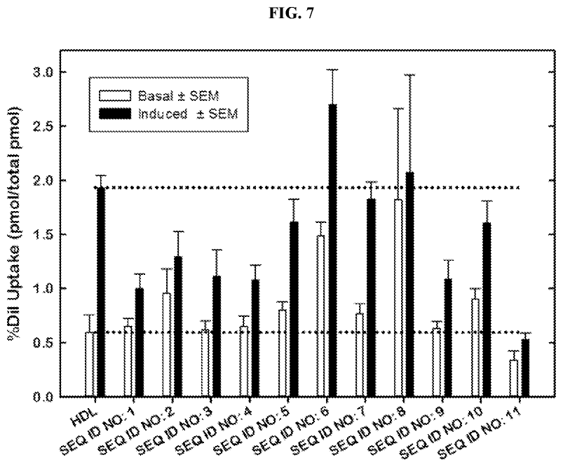

SR-BI interaction studies are done with BHK(SR-BI) cells obtained from the NIH. The cells were stably transfected with an inducible human SR-BI gene by means of the GeneSwitch™ System (Invitrogen). The cells were plated (96-well plate) (8000 cells/well) in growth medium (Dulbeco's modified Eagle medium containing 10% fetal bovine serum) containing 200 ug/ml each of zeocin and hygromycin. After 24 hours incubation, the growth medium was removed and replaced with 0.2% bovine serum albumin in Dulbeco's modified Eagle medium. The medium of cells to be induced for SR-BI expression also contained 10 nM mifepristone, added from a DMSO stock solution. DMSO alone was added to the medium of uninduced cells. The induction medium was removed after 24 hours incubation and replaced with medium containing DiI-labeled PALM (32 μg peptide/ml) or DiI-labeled HDL (19 μg protein/ml) (Kalen Biomedical, Montgomery Village, Md.). The test media were prepared by diluting an aliquot of Di-I-labeled PALM (Example 6) or the DiI-labeled HDL in 0.2% bovine serum albumin in Dulbeco's modified Eagle medium. The solutions were passed through 0.2 μm pore size, polyethersulfone, sterilization filters before use. The cells were incubated in test media for 4 hours. Next, the cells were washed 3 times with 0.1% albumin in Dulbeco's phosphate buffered saline (with calcium and magnesium). The last wash was replaced with 200 ul/well of t-butanol/water (95%/5%). The covered plate was left to stand at room temperature for 30 min with occasional shaking. The fluorescence in each well was detected at 520 nm excitation and 580 nm emission with a 550 nm cutoff filter on a Molecular Dynamics Gemini fluorescence plate reader, the results are shown in Table 6 and FIG. 7.

TABLE 6
SR-BI selectivity of peptide-amphiphile
lipid micelles as a function of peptide

| | Basal | Induced | SR-BI Dependent |
|---|---|---|---|
| Name | Uptake | Uptake ^{a} | Uptake ^{b} |
| HDL | 6 ± 2 | 19 ± 1 | 224% |
| SEQ ID NO: 1 | 6 ± 1 | 10 ± 1 | 53% |
| SEQ ID NO: 2 | 9 ± 2 | 12 ± 2 | 35% |
| SEQ ID NO: 3 | 6 ± 1 | 11 ± 2 | 79% |
| SEQ ID NO: 4 | 6 ± 1 | 10 ± 1 | 66% |
| SEQ ID NO: 5 | 8 ± 1 | 16 ± 2 | 104% |
| SEQ ID NO: 6 | 15 ± 1 | 27 ± 3 | 82% |
| SEQ ID NO: 7 | 8 ± 1 | 18 ± 2 | 137% |
| SEQ ID NO: 8 | 17 ± 8 | 20 ± 9 | 14% |
| SEQ ID NO: 9 | 6 ± 1 | 11 ± 2 | 72% |
| SEQ ID NO: 10 | 9 ± 1 | 16 ± 2 | 78% |
| SEQ ID NO: 11^{c} | 3 ± 1 | 5 ± 1 | 55% |

^{a} (DiI in cells)/(Total DiI added) × 10⁻³ ± SEM (n = 4)
^{b} Increase over basal uptake
^{c}LAP642 peptide, Homan et al. 2013

### Example 17 Effect of SR-BI Antibody on PALM(XT3) Cytotoxicity in SKOV-3 Cells

SKOV-3 were plated and incubated for 24 hour, as in Example 15. Next, growth medium was replaced with serum-free medium containing 0.5% albumin and the indicated concentrations of test agents, with or without anti-SRBI (1/250 dilution) (NB400-113, Novus Biologicals). The cells were incubated 12 hr. Next, the cells were washed with serum-free medium containing 0.5% albumin and grown a further 60 hour in growth medium. Cell growth was detected by MTT assay, as in Example 14 (FIG. 8).

### Example 18 SKOV-3 Ovarian Cell Xenograft Suppression in Nude Mice

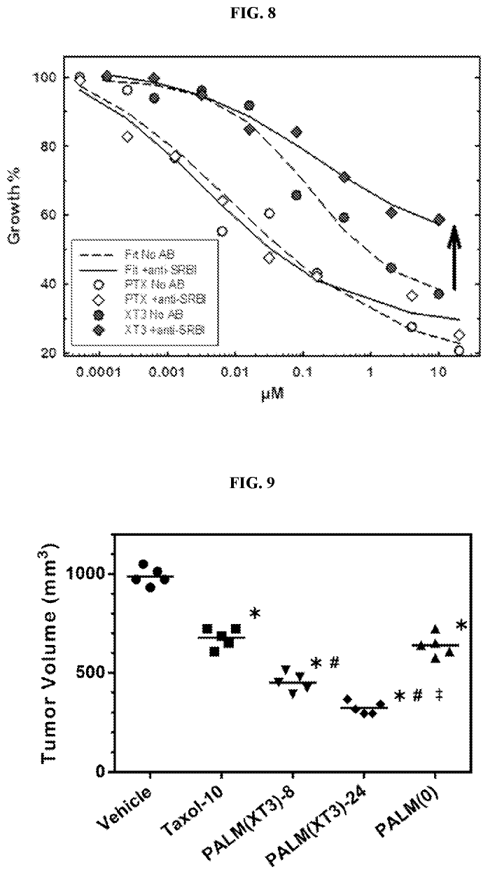

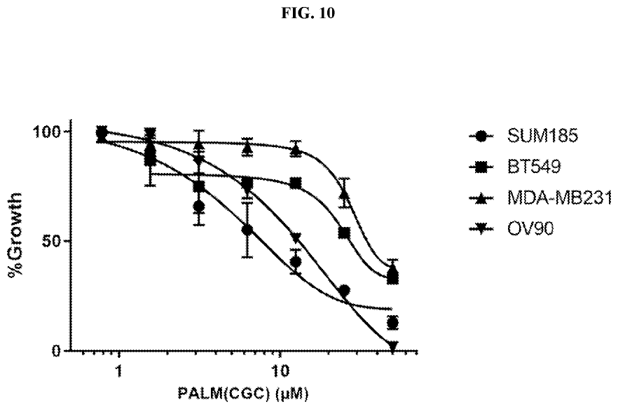

Female NU(NCr)-Foxn1nu nude mice (9-10 weeks old) were inoculated subcutaneously in the flank region with 0.1 ml of a 1:1 mixture of PBS and Matrigel containing 1×106 SKOV3 human ovarian tumor cells. When tumor volumes reached 50-100 mm3 in size (7 days after implantation), the mice were randomly assigned to five groups of 5 mice per group. Paclitaxel dosing solution was prepared to mimic Taxol®, the commercial paclitaxel formulation used to treat patients. Paclitaxel was dissolved at 6 mg/ml in a 1:1 mixture of Cremophor EL:ethanol, which was then diluted to 1.25 mg/ml with saline. PALM containing XT3 was prepared with SEQ ID NO:5 peptide by the protocol described in Example 10. Volumes were scaled up to meet the dosing needs. PALM dosing solutions were prepared containing 1 mg/ml PTX equivalents as XT3 and 3 mg/ml PTX equivalent as XT3, which corresponded to 5.8 mg/ml and 13.8 mg/ml SEQ ID NO:5 peptide, respectively. A third PALM solution for dosing, without XT3 added, was made at 17.3 mg/ml peptide. The vehicle control was 1 volume of Cremophor EL:ethanol (1:1) diluted with 4.8 volumes of saline. Mice were dosed via tail vein injection. Six total doses, each four days apart, were administered to each test group for the respective test solutions. The test groups consisted of: 1) vehicle, 2) 10 mg/kg paclitaxel, 3) 8 mg/kg paclitaxel equivalents of PALM, 4) 24 mg/kg paclitaxel equivalents of PALM, and 5) PALM at 138 mg/ml peptide, without XT. Tumor volumes were measured three times a week in two dimensions using an electronic caliper. Volume was calculated in mm3 using the formula: V=0.5 a×b² where a and b are the long and short diameters of the tumor, respectively. The final tumor volumes were recorded 35 days after start of dosing (FIG. 9).

### Example 19 δ-Tocotrienyl (N4)-Gemcitabine Carbamate

The hydroxyl groups in gemcitabine are protected by conversion to tert-butoxycarbonyl (BOC) esters with di-tert-butyl dicarbonate following the procedure of Guo and Gallo (J. Org. Chem. 1999, 64, 8319) to yield (6)

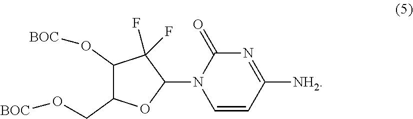 [SMILES: CC(C)(C)OC(=O)OCC1OC(n2ccc(N)nc2=O)C(F)(F)C1OC(=O)OC(C)(C)C]

Compound 5 is dissolved in anhydrous dichloromethane to a final concentration of 0.2M compound (5). For every mole of compound (5) in solution, 1.2 mole equivalents of compound (3) at 0.5M concentration in methylene chloride and 3 mole equivalents of DMAP are combined at rt. The mixture is stirred at room temperature for 24 h. The resultant product is deprotected with trifluoracetic acid, as referenced. Pure compound is obtained by flash column chromatography using dichloromethane and methanol eluent, beginning with 100% dichloromethane and gradually increasing the concentration to 10% methanol to yield the titled compound (6).

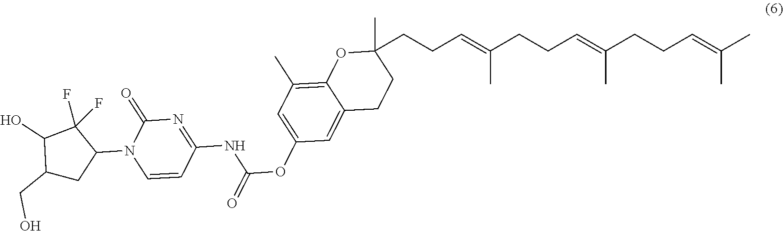 [SMILES: CC(C)=CCC/C(C)=C/CC/C(C)=C/CCC1(C)CCc2cc(OC(=O)Nc3ccn(C4CC(CO)C(O)C4(F)F)c(=O)n3)cc(C)c2O1]

### Example 20 Synthesis of (N4)-Gemcitabine Carbamates with the α, β or γ-Tocotrienol Isomers is Performed Similarly to Example 19

### Example 21 Cholesteryl (N4)-Gemcitabine Carbamate (CGC)

The synthesis of cholesteryl (N4)-gemcitabine carbamate (7) was performed in the same manner as described in Example 19 with the exception that compound (5) was reacted with cholesterol chloroformate (commercially available) and deprotected as in Example 19 to yield the titled compound (7).

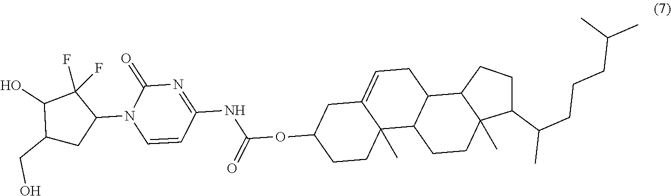 [SMILES: CC(C)CCCC(C)C1CCC2C3CC=C4CC(OC(=O)Nc5ccn(C6CC(CO)C(O)C6(F)F)c(=O)n5)CCC4(C)C3CCC12C]

### Example 22 Paclitaxel Linked to Fatty Alcohols Via Succinic and Diglycolic Acids

Synthesis of paclitaxel linked to fatty alcohol via a succinate or diglycolate di-ester link is accomplished by reacting fatty alcohol with 4-(dimethylamino)pyridine and succinic anhydride or diglycolic anhydride in anhydrous pyridine with constant stirring for 24 h at room temperature. The reaction is quenched with 0.1 N HCl in dichloromethane. The product is obtained by preparative TLC or flash column chromatography with ethyl acetate in petroleum ether. The alcohol-succinic acid or a diglycolic acid conjugate is combined with 4-(dimethylamino)pyridine and N-(3-dimethylaminopropyl)-N′-ethylcarbodiimide in dry dichloromethane. Paclitaxel is added into the reaction mixture. After 24 h, the reaction is quenched with water and extracted with dichloromethane. The product is obtained by preparative TLC using ethyl acetate/heptanes (50:50) as eluent.

### Example 23 Synthesis of 4-O-(Delta-Tocotrienyl Carbonate)-des-acetyl Vinblastine (8)

Vinblastine is deacetylated by the procedure of Brady et al, J. Med. Chem. 2002, 45, 4706-4715. Vinblastine sulfate (1.5 g) is dissolved in 100 mL of absolute methanol and combined with 30 ml of anhydrous hydrazine. The solution is stirred at room temperature for 16 hours. The solvent is removed by evaporation until a paste results. The product is partitioned with 200 ml methylene chloride and 100 ml saturated sodium bicarbonate. The bicarbonate phase is washed two times with 50 mL methylene chloride. Each of the methylene chloride phases isolated from the partioning is washed twice with 50 mL of water followed by one wash with 30 ml saturated NaCl. The organic phases are combined and dried over anhydrous Na₂SO₄. Solvent is removed by rotary evaporation. The remainder of the synthetic protocol follows essentially that described in Example 3, with exception that des-acetyl vinblastine is substituted for paclitaxel to yield the titled compound (8).

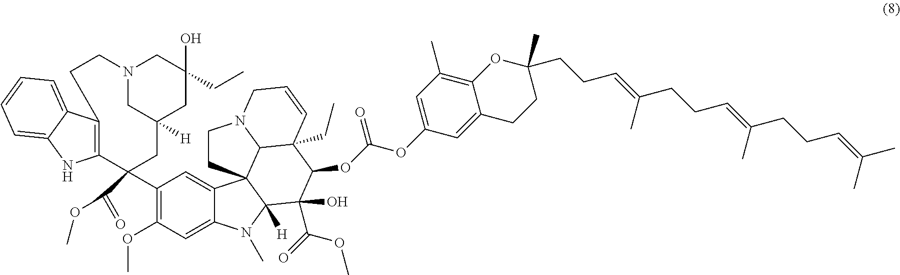

### Example 24 Growth Inhibition of Human Triple-Negative Breast Cancer (SUM185, BT549, MDA-MB231) and Ovarian Cancer Cells (0V90) by CGC in PALM

The preparation of PALM loaded with CGC essentially followed the protocol described in Example 10, substituting CGC for XT3 and using SEQ ID NO:5 peptide. Cells were grown and maintained in RPMI-1640 medium supplemented with 10% fetal bovine serum. The growth inhibition tests followed essentially the protocol described in Example 15. With the exception that the growth medium with highest concentration of CGC in PALM (50 μM) was diluted 50% with growth medium to produce the next lower concentration test solution (25 μM). This process was repeated until seven test solutions of growth medium ranging from 50 μM to 0.78 μM CGC were produced. Test solutions, in 100 μL aliquots, were added to cells growing in 96-well plates. Each test solution was delivered to three wells. Cells were incubated 48 hr and then subjected to the MTT assay. The growth inhibition tests follow essentially as the protocol described in Example 14.

While a number of embodiments of this disclosure are described, it is apparent that the basic examples may be altered to provide other embodiments that use or encompass methods and processes of this invention. The embodiments and examples are for illustrative purposes and are not to be interpreted as limiting the disclosure, but rather, the appended claims define the scope of this invention.

1. A peptide comprising an amino acid sequence: X1-X2-X3-X4-X5-X6-X7-X8-X9-X10-X11-X12-X13-X14-X15-X16-X17-X18-X19-X20, wherein: X1 is D; X2 is V, Aib, Amy, or Aml; X3 and X10 are each F; X4 and X19 are each Q; X5, X16, and X18 are each K; X6, X9, and X13 are each L; X7 and X14 are each independently selected from the group consisting of Aib, Amy, Aml, Amp, Amt; X8 and X15 are each E; X11 and X12 are each independently selected from the group consisting of Q and N; X17 is W; and X20 is V, (SEQ ID NO:12) wherein the peptide is optionally acylated at the N-terminus, amidated at the C-terminus, or both acylated at the N-terminus and amidated at the C-terminus and the peptide is from 20 to 24 amino acids in length.

2. The peptide of claim 1, wherein the peptide is 20 amino acids in length.

3. A peptide comprising an amino acid sequence selected from the group consisting of SEQ ID NO:7, SEQ ID NO:9, or SEQ ID NO:10, wherein the peptide is optionally acylated at the N-terminus, amidated at the C-terminus, or both acylated at the N-terminus and amidated at the C-terminus and the peptide is from 20 to 24 amino acid in length.

4. The peptide according to claim 3, wherein the amino acid consists essentially of the amino acid sequence SEQ ID NO:7, SEQ ID NO:9, or SEQ ID NO:10, wherein the peptide is optionally acylated at the N-terminus, amidated at the C-terminus, or both acylated at the N-terminus and amidated at the C-terminus and the peptide is from 20 to 24 amino acid in length.

5. The peptide according to claim 3, consisting of the amino acid sequence SEQ ID NO:7, SEQ ID NO:9, or SEQ ID NO:10, wherein the peptide is optionally acylated at the N-terminus, amidated at the C-terminus, or both acylated at the N-terminus and amidated at the C-terminus and the peptide is 20 amino acid in length.

6. The peptide according to claim 3, consisting of the amino acid sequence SEQ ID NO:7, SEQ ID NO:9, or SEQ ID NO:10, wherein the peptide is not acylated or amidated.

7. The peptide according to claim 6, wherein the amino acid sequence is SEQ ID NO:7.

8. A peptide-amphiphile lipid micelle (PALM) comprising a peptide according to claim 1, and a lipid component comprising sphingomyelin and one or more additional phospholipids.

9. The PALM according to claim 8, wherein the one or more additional phospholipid is selected from the group consisting of phosphatidylcholine, polyethylene glycol-phosphatidylethanolamine (PEG-PE), phosphatidylethanolamine, phosphatidylglycerol, phosphatidylserine, phosphatidylinositol, cardiolipin, or any combination thereof.

10. The PALM according to claim 8, wherein the one or more additional phospholipid comprises a phosphatidylcholine.

11. The PALM according to claim 10, wherein the phosphatidylcholine is 1-palmitoyl-2-oleoyl-phosphatidylcholine (POPC).

12. The PALM according to claim 8, wherein the molar ratio of phospholipid to sphingomyelin is from about 80:20 to about 60:40.

13. The PALM according to claim 12, wherein the molar ratio of phospholipid to sphingomyelin is about 70:30.

14. The PALM according to claim 8, wherein the molar ratio of the lipid component to peptide is from about 10:1 to about 2:1.

15. The PALM according to claim 13, wherein the molar ratio of the lipid component to peptide is from about 6:1 to about 4:1.

16. The PALM according to claim 8, wherein the PALM has a mean particle diameter is from about 7.5 nm to about 20 nm.

17. A PALM-cargo composition comprising the PALM according to claim 8, and at least one cargo molecule.

18. The PALM-cargo composition according to claim 17, wherein the at least one cargo molecule is a compound conjugate having the formula (I):
A-R-L-X (formula I)
wherein A is an agent having a hydroxyl or an amine group; R is the hydroxyl group or the amine group of the agent; L is a linker; and X is an anchor moiety.

19. The PALM-cargo composition of claim 18, wherein the anchor moiety X is cholesterol, α-tocotrienol, β-tocotrienol, γ-tocotrienol, and δ-tocotrienol, cholecalciferol, or ergocalciferol.

20. The PALM-cargo composition of claim 19, wherein L is diglycolic acid, carbonic acid, or succinic acid.

21. The PALM-cargo composition of claim 19, wherein R is the hydroxyl group and the anchor moiety is covalently bonded to the agent by a carbonate ester bond, or a succinic ester bond, or a diglycolic ester bond.

22. The PALM-cargo composition of claim 19, wherein R is an amine group and the anchor moiety is covalently bonded to the agent by a carbamate ester bond, or a succinic ester bond, or a diglycolic ester bond.

23. The PALM-cargo composition according to claim 18, wherein the agent is a chemotherapeutic agent.

24. The PALM-cargo composition according to claim 23, wherein the chemotherapeutic agent is selected from the group consisting of adenosine, AZD2811, bortezomib, 10-hydroxy camptothecin, 7-ethyl-10-hydroxycamptothecin, daunorubicin, docetaxel, doxorubicin, eribulin, gemcitabine, ixabepilone, mertansine, miriplatin, misonidazole, paclitaxel, topotecan, tubulysin A, vincristine, vinblastine, vinorelbine, and combinations thereof.

25. The PALM-cargo composition according to claim 24, wherein the chemotherapeutic agent is 10-hydroxy camptothecin, daunorubicin, doxorubicin, gemcitabine, topotecan, paclitaxel, or docetaxel.

26. The PALM-cargo composition according to claim 24, wherein the compound conjugate is paclitaxel 2′-□-tocotrienyl carbonate; paclitaxel 2′-cholesteryl carbonate; docetaxel 2′-cholesteryl carbonate; daunorubicin □-tocotrienyl carbonate; gemcitabine cholesteryl carbonate; 10-hydroxycampothecin cholesterol carbonate, or 7-ethyl-10-hydroxycamptothecin cholesteryl carbonate.

27. The PALM-cargo composition according to claim 18, wherein the PALM-cargo composition further comprises an imaging agent.

28. The PALM-cargo composition according to claim 27, wherein the imaging agent is 1,2-dipalmitoyl-sn-glycero-3-phosphoethanolamine-N-diethylenetriaminepentaacetic acid (gadolinium salt) (PE-DTPA(Gd)), 1,2-dipalmitoyl-sn-glycero-3-phosphoethanolamine-N-diethylenetriaminepentaacetic acid (manganese salt) (PE-DTPA(Mn)), ¹¹¹In-DTPA-A (indium salt).

29. The PALM-cargo composition according to claim 18, wherein the peptide is SEQ ID NO:7, SEQ ID NO:9, or SEQ ID NO:10.

30. The PALM-cargo composition according to claim 18, wherein the peptide is SEQ ID NO:7.

31. A method for treating a disorder comprising administering to a subject in need thereof, an effective amount of a PALM-cargo composition according to claim 18.

32. The method for treating a disorder according to claim 31, wherein the peptide in the PALM-cargo composition is SEQ ID NO:7, SEQ ID NO:9, or SEQ ID NO:10.

33. The method for treating a disorder according to claim 32, wherein the peptide in the PALM-cargo composition is SEQ ID NO:7.

34. The method for treating a disorder according to claim 32, wherein the drug is a chemotherapeutic agent.

35. The method for treating a disorder according to claim 34, wherein the chemotherapeutic agent in the PALM-cargo composition is 10-hydroxy camptothecin, daunorubicin, doxorubicin, gemcitabine, topotecan, paclitaxel, or docetaxel.

36. The method according to claim 31, wherein the disorder is cancer.

37. The method according to claim 36, wherein the cancer is ovarian cancer, cervical cancer, colorectal cancer, prostate cancer, breast cancer, gastric adenocarcinoma, head and neck cancer, testicular cancer, leukemia, neuroblastoma, Hodgkin's lymphoma, non-Hodgkin's lymphoma, and non-small cell lung cancer.


SEQUENCE LISTING (12 sequences)

SEQ ID NO: 1 - protein, 20 aa, Artificial Sequence
  synthetic peptide
  MOD_RES (7)..(7): Aib
DVFQKLXELFNQLLEKWKQV

SEQ ID NO: 2 - protein, 20 aa, Artificial Sequence
  synthetic peptide
DVFQKLVELFNQLLEKWKQV

SEQ ID NO: 3 - protein, 20 aa, Artificial Sequence
  synthetic peptide
  MOD_RES (3)..(3): Aib
DVXQKLFELFNQLLEKWKQV

SEQ ID NO: 4 - protein, 20 aa, Artificial Sequence
  synthetic peptide
  MOD_RES (3)..(3): Aib
DVXQKFLELFNQLLEKWKQV

SEQ ID NO: 5 - protein, 20 aa, Artificial Sequence
  synthetic peptide
  MOD_RES (7)..(7): Aib
  MOD_RES (14)..(14): Aib
DVFQKLXELFNQLXEKWKQV

SEQ ID NO: 6 - protein, 20 aa, Artificial Sequence
  synthetic peptide
  MOD_RES (7)..(7): Aib
  MOD_RES (14)..(14): Aib
DVFQKLXELFNQLXEKFKQV

SEQ ID NO: 7 - protein, 20 aa, Artificial Sequence
  synthetic peptide
  MOD_RES (2)..(2): Aib
  MOD_RES (7)..(7): Aib
  MOD_RES (14)..(14): Aib
DXFQKLXELFNQLXEKWKQV

SEQ ID NO: 8 - protein, 20 aa, Artificial Sequence
  synthetic peptide
  MOD_RES (7)..(7): Aib
DVFQKLXELFKELLEKWKQV

SEQ ID NO: 9 - protein, 20 aa, Artificial Sequence
  synthetic peptide
  MOD_RES (7)..(7): Aib
  MOD_RES (14)..(14): Aib
DVFQKLXELFQQLXEKWKQV

SEQ ID NO: 10 - protein, 20 aa, Artificial Sequence
  synthetic peptide
  MOD_RES (7)..(7): Aib
  MOD_RES (14)..(14): Aib
DVFQKLXELFQNLXEKWKQV

SEQ ID NO: 11 - protein, 22 aa, Artificial Sequence
  synthetic peptide
PVLDLFRELLNELLEALKQKLK

SEQ ID NO: 12 - protein, 20 aa, Artificial Sequence
  synthetic peptide
  MOD_RES (2)..(2): V
  MOD_RES (2)..(2): Aib
  MOD_RES (2)..(2): Amv
  MOD_RES (2)..(2): Aml
  MOD_RES (7)..(7): Aib
  MOD_RES (7)..(7): Aml
  MOD_RES (7)..(7): Amp
  MOD_RES (7)..(7): Amt
  MOD_RES (7)..(7): amv
  MOD_RES (11)..(11): Q
  MOD_RES (11)..(11): N
  MOD_RES (12)..(12): N
  MOD_RES (12)..(12): Q
  MOD_RES (14)..(14): Aib
  MOD_RES (14)..(14): Amv
  MOD_RES (14)..(14): Aml
  MOD_RES (14)..(14): Amp
  MOD_RES (14)..(14): Amt
DXFQKLXELFXXLXEKWKQV